SEQUENCE LISTING (32 sequences)

SEQ ID NO: 1 - protein, 560 aa, Artificial Sequence
  Bifidobacterium moukalabense maltotriose amylase with Bacillus licheniformis keratinase signal peptide
  MISC_FEATURE (1)..(560): Bifidobacterium moukalabense maltotriose amylase with Bacillus licheniformis keratinase signal peptide
MMRKKSFWLGMLTAFMLVFTMAFSDSASAQPEARTSAKKDVQVIAFQQSWNTIAKECTDTYGPEGVAYVEVSPPQESIQGTQWWTSYQPVSYKLDSKLGTEAEFKNMIKQCNAAGVDIIADVVINQTTGSDVAAGDQKGVAGSDYNGSTGTYPGFATSQYPGGITDSDFHGCKRNISDYTNQQEVQECRLSSMWDFDSESEKVQDIQSDYLVSLWNAGVRAFRMDAVKHINTNSMKALKEKFAQKIGKNANDIYWIQEVIGNSSEASGIQPSNYLQNGTVTQFGFKSEMNQAFKDKIAKLKGLKDRLDKDIDSANANVFVTNWDTARNQGALTYKDGSRFQLANAFMLAYDYGTPRLLSDYKWDVNDNGAPGATDTKVPDVDMGSACSANSSDWNCEQRWTSTRGMIAFHNYAGDAAVTDWQDDGDSNIAFSRDGKGFIAINNSKDDKNASYTTSLADGEYCDVYAVMDCSKTVTVKGGKVETKIPAHSAIALYAGATKTSHPQASAAKDPSDPDIGQQDDEVSATDKTVTIYYKPANATGRPPRSTTVWAMTGTSRKPT

SEQ ID NO: 2 - protein, 560 aa, Artificial Sequence
  maltotriose amylase variant
  MISC_FEATURE (1)..(560): maltotriose amylase variant
MMRKKSFWLGMLTAFMLVFTMAFSDSASAQPEARTSAKKDVQVIAFQQSWNTIAKECTDTYGPEGVAYVEVSPPQESIQGTQWWTSYQPVSYKLDSKLGTEAEFKNMIKQCNAAGVDIIADVVINQTTGSDVAAGDQKGVAGSDYNGSTGTYPGFATSQYPGGITDSDFHGCKRNISDYTDQQEVQECRLSSMWDFDSESEKVQDIQSDYLVSLWNAGVRAFRMDAVKHINTNSMKALKEKFAQKIGKNANDIYWIQEVIGNSSEASGIQPSNYLQNGTVTQFGFKSEMNQAFKDKRAKLKGLKDRLDKDIDSANANVFVTNWDTARNQGALTYKDGSRFQLANAFMLAYDYGTPRLLSDYKWDVRDNGAPGATDTKVPDVDMGSACSANSSDWNCEQRWTSTRGMIAFHNYAGDAAVTDWQDDGDSNIAFSRDGKGFIAINNSKDDKNASYTTSLADGEYCDVYAVMDCSKTVTVKGGKVETKIPAHSAIALYAGATKTSHPQASAAKDPSDPDIGQQDDEVSATDKTVTIYYKPANATGRPPRSTTVWAMTGTSRKPT

SEQ ID NO: 3 - protein, 560 aa, Artificial Sequence
  maltotriose amylase variant
MMRKKSFWLGMLTAFMLVFTMAFSDSASAQPEARTSAKKDVQVILFQQNWNTIAKECTDTYGPEGVAYVEVSPPQESIQGTQWWTSYQPVSYKLDSKLGTEAEFKNMIKQCNAAGVDIIADVVINQTTGSDVAAGDQKGVAGSDYNGSTGTYPGFATSQYPGGITDSDFHGCKRNISDYTNQQEVQECRLSSMWDFDSESEKVQDIQSDYLVSLWNAGVRAFRMDAVKHINTNSMKALKEKFAQKIGKNANDIYWIQEVIGNSSEASGIQPSNYLQNGTVTQFGFKSEMNQAFNDKIAKLKGLKDRLDKDIDSANANVFVTNWDTARNQGALTYKDGSRFQLANAFMLAYDYGTPRLLSDYKWDVNDNGAPGATDTKVPDVDMGSACSANSSDWNCEQRWTSTRGMIAFHNYAGDAAVTDWQDDGDSNIAFSRDGKGFIAINNSKDDKNASYTTSLADGEYCDVYAVMDCSKTVTVKGGKVETKIPAHSAIALYAGATKTSHPQASAAKDPSDPDIGQQDDEVSATDKTVTIYYKPANATGRPPRSTTVWAMTGTSRKPT

SEQ ID NO: 4 - protein, 560 aa, Artificial Sequence
  maltotriose amylase variant
MMRKKSFWLGMLTAFMLVFTMAFSDSASAQPEARTSAKKDVQVIAFQQSWNTIAKECTDTYGPEGVAYVEVSPPQESIQGTQWWTSYQPVSYKLDSKLGTEAEFKNMIKQCNAAGVDIIADVVINHTTGSDVAAGDQKGVAGSDYNGSTGTYPGFATSQYPGGITDSDFHGCKRNISDYTNQQEVQECRLSSMWDFDSESEKVQDIQSDYLVSLWNAGVRAFRMDAVKHINTNSMKALKEKFAQKIGKNANDIYWIQEVIGNSSEASGIQPSNYLQNGTVTQFGFKSEMNQAFKDKIAKLKGLKDRLDKDIDSANANVFVTNWDTARNQGALTYKDGSRFQLANAFMLAYDYGTPRLLSDYKWDVNDNGAPNATDTKVPDVDMGNACSANSSDWNCEQRWTSTRGMIAFHNYAGDAAVTDWQDDGDSNIAFSRDGKGFIAINNSKDDKNASYTTSLADGEYCDVYAVMDCSKTVTVKGGKVETKIPAHSAIALYAGATKTSHPQASAAKDPSDPDIGQQDDEVSATDKTVTIYYKPANATGRPPRSTTVWAMTGTSRKPT

SEQ ID NO: 5 - protein, 560 aa, Artificial Sequence
  maltotriose amylase variant
MMRKKSFWLGMLTAFMLVFTMAFSDSASAQPEARTSAKKDVQVIAFQQNWNTIAKECTDTYGPEGVAYVEVSPPQESIQGTQWWTSYQPVSYKLDSKLGTEAEFKNMIKQCNAAGVDIIADVVINQTTGSDVAAGDQKGVAGSDYNGSTGTYPGFATSQYPGGITDSDFHGCKRNISDYTNQQEVQECRLSSMWDFDSESEKVQDIQSDYLVSLWNAGVRAFRMDAVKHINTNSMKALKEKFAQKIGKNANDIYWIQEVIGNSSEASGIQPSNYLQNGTVTQFGYKSEMNQAFKDKIAKLKGLKDRLDKDIDSANANVFVTNWDTARNQGALTYKDGSRFQLANAFMLAYDYGTPRLLSDYKWDVNDNGAPGATDTKVPDVDMGSACSSNSSDWNCEQRWTSTRGMIAFHNYAGDAAVTDWQDDGDSNIAFSRDGKGFIAINNSKDDKNASYTTSLADGEYCDVYAVMDCSKTVTVKGGKVETKIPAHSAIALYAGATKTSHPQASAAKDPSDPDIGQQDDEVSATDKTVTIYYKPANATGRPPRSTTVWAMTGTSRKPT

SEQ ID NO: 6 - protein, 560 aa, Artificial Sequence
  maltotriose amylase variant
MMRKKSFWLGMLTAFMLVFTMAFSDSASAQPEARTSAKKDVQVIAFQQSWNTIAKECTDTYGPEGVAYVEVSPPQESIQGTQWWTSYQPVSYKLDSKLGTEAEFKNMIKQCNAAGVDIIADVVINQTTGSDVAAGDGKGVAGSDYNGSDGTYPGFATSQYPGGITDSDFHGCKRNISDYTNQQEVQECRLSSMWDFDSESEKVQDIQSDYLVSLWNAGVRAFRMDAVKHINTNSMKALKEKFAQKIGKNANDIYWIQEVIGNSSEASGIQPSNYLQNGTVTQFGFKSEMNQAFKDKIAKLKGLKDRLDKDIDSANANVFVTNWDTARNQGALTYKDGSRFQLANAFMLAYDYGTPRLLSDYKWDVADNGAPGATDTKVPDVDMGSACSANSSDWNCEQRWTSTRGMIAFHNYAGDAAVTDWQDDGDSNIAFSRDGKGFIAINNSKDDKNASYTTSLADGEYCDVYAVMDCSKTVTVKGGKVETKIPAHSAIALYAGATKTSHPQASAAKDPSDPDIGQQDDEVSATDKTVTIYYKPANATGRPPRSTTVWAMTGTSRKPT

SEQ ID NO: 7 - protein, 560 aa, Artificial Sequence
  maltotriose amylase variant
MMRKKSFWLGMLTAFMLVFTMAFSDSASAQPEARTSAKKDVQVIAFQQSWNTIAKECTDTYGPEGVAYVEVSPPQESIQGTQWWTSYQPVSYKLDSKLGTEAEFKNMIKQCNAAGVDIIADVVINQTTGSDVAAGDQKGVAGSDYNGSTGTYPGFATSQYPGGIQDSDFHGCKRNISDYTNQQEVQECRLSSMWDFDSESEKVQDIQSDYLVSLWNAGVRAFRMDAVKHINTNSMKALKEKFAQKIGKNANDIYWIQEVIGNSSEASGIQPSNYLQNGTVTQFGFKSEMNQAFNDKIAKLKGLKDRLDKDIDSAKANVFVTNWDTARNQGALTYKDGSRFQLANAFMLAYDYGTPRLLSDYKWDVNDNGAPGATDTKVPDVDMGSACSANSSDWNCEQRWTSTRGMIAFHNYAGDAAVTDWQDDGDSNIAFSRDGKGFIAINNSKDDKNASYTTSLADGEYCDVYAVMDCSKTVTVKGGKVETKIPAHSAIALYAGATKTSHPQASAAKDPSDPDIGQQDDEVSATDKTVTIYYKPANATGRPPRSTTVWAMTGTSRKPT

SEQ ID NO: 8 - protein, 560 aa, Artificial Sequence
  maltotriose amylase variant
MMRKKSFWLGMLTAFMLVFTMAFSDSASAQPEARTSAKKDVQVIAFQQSWNTIAKECTDTYGPEGVAYVEVSPPQESIQGTQWWTSYQPVSYKLDSKLGTEAEFKNMIKQCNAAGVDIIADVVINQTTGSDVAAGDQKGVAGSDYNGSTGTYPGFATSQYPGGITDSDFHGCKRNISDYTNQQEVQECRLSSMWDFDSESEKVQDIQSDYLVSLWNAGVRAFRMDAVKHINTNSMKALKEKFAQKIGKNANDIYWIQEVIGNSSEASGIQPSNYLQNGTVTQFGFKSEMNQAFNDKIAKLKGLKDRLDKDIDSAQANVFVTNWDTARNQGALTYKDGSRFQLANAFMLAYDYGTPRLLSDYKWDVNDNGAPGATDTKVPDVDMGSACSADSSDWNCEQRWTSTRGMIAFHNYAGDAAVTDWQDDGDSNIAFSRDGKGFIAINNSKDDKNASYTTSLADGEYCDVYAVMDCSKTVTVKGGKVETKIPAHSAIALYAGATKTSHPQASAAKDPSDPDIGQQDDEVSATDKTVTIYYKPANATGRPPRSTTVWAMTGTSRKPT

SEQ ID NO: 9 - protein, 560 aa, Artificial Sequence
  maltotriose amylase variant
MMRKKSFWLGMLTAFMLVFTMAFSDAASAQPEARTSAKKDVQVIAFQQNWATIAKECTDTYGPEGVAYVEVSPPQESIQGTQWWTSYQPVSYKLDSKLGTEAEFKNMIKQCNAAGVDIIADVVINQTTGSDVAAGDQKGVAGSDYNGSTGTYPGFATSQYPGGITDSDFHGCKRNISDYTNQQEVQECRLSSMWDFDSESEKVQDIQSDYLVSLWNAGVRAFRMDAVKHINTNSMKALKEKFAQKIGKNANDIYWIQEVIGNSSEASGIQPSNYLQNGTVTQFGFKSEMNQAFKDKIAKLKGLKDRLDKDIDSANANVFVTNWDTARNQGALTYKDGSRFQLANAFMLAYDYGTPRLLSDYKWDVNDNGAPGATDTKVPDVDMGSACSANSSDWNCEQRWTSTRGMIAFHNYAGDAAVTDWQDDGDSNIAFSRDGKGFIAINNSKDDKNASYTTSLADGEYCDVYAVMDCSKTVTVKGGKVETKIPAHSAIALYAGATKTSHPQASAAKDPSDPDIGQQDDEVSATDKTVTIYYKPANATGRPPRSTTVWAMTGTSRKPT

SEQ ID NO: 10 - protein, 560 aa, Artificial Sequence
  maltotriose amylase variant
MMRKKSFWLGMLTAFMLVFTMAFSDSASAQPEARTSAKKDVQVIAFQQNWNTIAKECTDTYGPEGVAYVEVSPPQESIQGTQWWTSYQPVSYKLDSKLGTEAEFKNMIKQCNAAGVDIIADVVINQTTGSDVAAGDQKGVAGSDYNGSTGTYPGFATSQYPGGITDSDFHDCKRNISDYTNQQEVQECRLSSMWDFDSESEKVQDIQSDYLVSLWNAGVRAFRMDAVKHINTNSMKALKEKFAQKIGKNANDIYWIQEVIGNSSEASGIQPSNYLQNGTVTQFGFKSEMNQAFNDKIAKLKGLKDRLDKDIDSAQANVFVTNWDTARNQGALTYKDGSRFQLANAFMLAYDYGTPRLLSDYKWDVNDNGAPGATDTKVPDVDMGNACSADSSDWNCEQRWTSTRGMIAFHNYAGDAAVTDWQDDGDSNIAFSRDGKGFIAINNSKDDKNASYTTSLADGEYCDVYAVMDCSKTVTVKGGKVETKIPAHSAIALYAGATKTSHPQASAAKDPSDPDIGQQDDEVSATDKTVTIYYKPANATGRPPRSTTVWAMTGTSRKPT

SEQ ID NO: 11 - protein, 560 aa, Artificial Sequence
  maltotriose amylase variant
MMRKKSFWLGMLTAFMLVFTMAFSDSASAQPEARTSAKKDVQVIAFQQSWNTIAKECTDTYGPEGVGYVEVSPPQESIQGTQWWTSYQPVSYKLDSKLGTEAEFKNMIKQCNAAGVDIIADVVINHTTGSDVAAGDQKGVAGSDYNGSTGTYPGFATSQYPGGITDSDFHGCKRNISDYTNQQEVQECRLSSMWDFDSESEKVQDIQSDYLVSLWNAGVRAFRMDAVKHINTNSMKALKEKFAQKIGKNANDIYWIQEVIGNSSEASGIQPSNYLQNGTVTQFGFKSEMNQAFNDKIAKLKGLKDRLDKDIDSAQANVFVTNWDTARNQGALTYKDGSRFQLANAFMLAYDYGTPRLLSDYKWDVNDNGAPGATDTKVPDVDMGSACSANSSDWNCEQRWTSTRGMIAFHNYAGDAAVTDWQDDGDSNIAFSRDGKGFIAINNSKDDKNASYTTSLADGEYCDVYAVMDCSKTVTVKGGKVETKIPAHSAIALYAGATKTSHPQASAAKDPSDPDIGQQDDEVSATDKTVTIYYKPANATGRPPRSTTVWAMTGTSRKPT

SEQ ID NO: 12 - protein, 560 aa, Artificial Sequence
  maltotriose amylase variant
MMRKKSFWLGMLTAFMLVFTMAFSDSASAQPEARTSAKKDVQVIAFQQSWNTIAKECTDTYGPEGVAYVEVSPPQESIQGTQWWTSYQPVSYKLDSKLGTEAEFKNMIKQCNAAGVDIIADVVINHTTGSDVAAGDQKGVAGSDYNGSTGTYPGFATSQYPGGITDSDFHGCKRNISDYTNQQEVQECRLSSMWDFDSESEKVQDIQSDYLVSLWNAGVRAFRMDAVKHINTNSMKALKEKFAQKIGKNANDIYWIQEVIGNSSEASGIQPSNYLQNGTVTQFGFKSEMNQAFNDKIAKLKGLKDRLDKAIDSAQANVFVTNWDTARNQGALTYKDGSRFQLANAFMLAYDYGTPRLLSDYKWDVNDNGAPGATDTKVPDVDMGSACSSDSSDWNCEQRWTSTRGMIAFHNYAGDAAVTDWQDDGDSNIAFSRDGKGFIAINNSKDDKNASYTTSLADGEYCDVYAVMDCSKTVTVKGGKVETKIPAHSAIALYAGATKTSHPQASAAKDPSDPDIGQQDDEVSATDKTVTIYYKPANATGRPPRSTTVWAMTGTSRKPT

SEQ ID NO: 13 - protein, 560 aa, Artificial Sequence
  maltotriose amylase variant
MMRKKSFWLGMLTAFMLVFTMAFSDSASAQPEARTSAKKDVQVIAFQQSWNTIAKECTDTYGPEGVAYVEVSPPQESIQGTQWWTSYQPVSYKLDSKLGTEAEFKNMVKQCNAAGVDIIADVVINQTTGSDVAAGDQKGVAGSDYNGSTGTYPGFATSQYPGGITDSDFHGCKRNISDYTNQQEVQECRLSSMWDFDSESEKVQDIQSDYLVSLWNAGVRAFRMDAVKHINTNSMKALKEKFAQKIGKNANDIYWIQEVIGNSSEASGIQPSNYLQNGTVTQFGFKSEMNQAFNDKIAKLKGLKDRLDKDIDSAQANVFVTNWDTARNQGALTYKDGSKFQLANAFMLAYDYGTPRLLSDYKWDVNDNGAPGATDTKVPDVDMGSACSANSSDWNCEQRWTSTRGMIAFHNYAGDAAVTDWQDDGDSNIAFSRDGKGFIAINNSKDDKNASYTTSLADGEYCDVYAVMDCSKTVTVKGGKVETKIPAHSAIALYAGATKTSHPQASAAKDPSDPDIGQQDDEVSATDKTVTIYYKPANATGRPPRSTTVWAMTGTSRKPT

SEQ ID NO: 14 - protein, 560 aa, Artificial Sequence
  maltotriose amylase variant
MMRKKSFWLGMLTAFMLVFTMAFSDSASAQPEARTSAKKDVQVIAFQQSWNTIAKECTDTYGPEGVAYVEVSPPQESIQGTQWWTSYQPVSYKLDSKLGTEAEFKNMIKQCNAAGVDIIADVVINHTTGSDVAAGDQKGVAGSDYNGSTGTYPGFATSQYPGGITDSDFHGCKRNISDYTNQQEVQECRLSSMWDFDSESEKVQDIQSDYLVSLWNAGVRAFRMDAVKHINTNSMKALKEKFAQKIGKNANDIYWIQEVIGNASEASGIQPSNYLQNGTVTQFGFKSEMNQAFNDKIAKLKGLKDRLDKDIDSAQANVFVTNWDTARNQGALTYKDGSRFQLANAFMLAYDYGTPRLLSDYKWDNNDNGAPGATDTKVPDVDMGSACSADSSDWNCEQRWTSTRGMIAFHNYAGDAAVTDWQDDGDSNIAFSRDGKGFIAINNSKDDKNASYTTSLADGEYCDVYAVMDCSKTVTVKGGKVETKIPAHSAIALYAGATKTSHPQASAAKDPSDPDIGQQDDEVSATDKTVTIYYKPANATGRPPRSTTVWAMTGTSRKPT

SEQ ID NO: 15 - protein, 560 aa, Artificial Sequence
  maltotriose amylase variant
MMRKKSFWLGMLTAFMLVFTMAFSDSASAQPEARTSAKKDVQVIAFQQSWNTIAKECTDTYGPEGVAYVEVSPPQESIQGTQWWTSYQPVSYKLDSKLGTEAEFKNMIKQCNAAGVDIIADVVINHTTGSDVAAGDQKGVAGSDYNGSDGTYPGFATSQYPGGITDSDFHGCKRNISDYTNQQEVQECRLSSMWDFDSESDKVQDIQSDYLVSLWNAGVRAFRMDAVKHINTNSMKALKEKFAQKIGKNANDIYWIQEVIGNSSEASGIQPSNYLQNGTVTQFGFKSEMNQAFNDKIAKLKGLKDRLDKDIDSAQANVFVTNWDTARNQGALTYKDGSRFQLANAFMLAYDYGTPRLLSDYKWDVNDNGAPGATDTKVPDVDMGSACSADSSDWNCEQRWTSTRGMIAFHNYAGDAAVTDWQDDGDSNIAFSRDGKGFIAINNSKDDKNASYTTSLADGEYCDVYAVMDCSKTVTVKGGKVETKIPAHSAIALYAGATKTSHPQASAAKDPSDPDIGQQDDEVSATDKTVTIYYKPANATGRPPRSTTVWAMTGTSRKPT

SEQ ID NO: 16 - protein, 560 aa, Artificial Sequence
  maltotriose amylase variant
MMRKKSFWLGMLTAFMLVFTMAFSDSASAQPEARTSAKKDVQVIAFQQSWNTIAKECTDTYGPEGVAYVEVSPPQESIQGTQWWTSYQPVSYKLDSKLGTEAEFKNMIKQCNAAGVDIIADVVINHTTGSDVAAGDQKGVAGSDYNGSTGTYPGFATSQYPGGITDSDFHGCKRNISDYTNQQEVQECRLSSMWDFDSESEKVQDIQSDYLVSLWNAGVRAFRMDAVKHINTNSMKALKEKFAQKIGKNANDIYWIQEVIGNSSEASGIQPGNYLQNGTVTQFGFKSEMNQAFNDKIAKLKGLKDRLDKDIDSAQANVFVTNWDTARNQGALSYKDGSRFQLANAFMLAYDYGTPRLLSDYKWDVNDNGAPGATDTKVPDVDMGSACSADSSDWNCEQRWTSTRGMIAFHNYAGDAAVTDWQDDGDSNIAFSRDGKGFIAINNSKDDKNASYTTSLADGEYCDVYAVMDCSKTVTVKGGKVETKIPAHSAIALYAGATKTSHPQASAAKDPSDPDIGQQDDEVSATDKTVTIYYKPANATGRPPRSTTVWAMTGTSRKPT

SEQ ID NO: 17 - protein, 560 aa, Artificial Sequence
  maltotriose amylase variant
MMRKKSFWLGMLTAFMLVFTMAFSDSASAQPEARTSAKKDVQVIAFQQSWNTIAKECTDTYGPEGVGYVEVSPPQESIQGTQWWTSYQPVSYKLASKLGTEAEFKNMVKQCNAAGVDIIADVVINHTTGSDVAAGDQKGVAGSDYNGSTGTYPGFATSQYPGGITDSDFHDCKRNISDYTNQQEVQECRLSSMWDFDTESEKVQDIQSDYLVSLWNAGVRAFRMDAVKHINTNSMKALKEKFAQKIGKNANDIYWIQEVIGNSSEASGIQPSNYLQNGTVTQFGFKSEMNQAFNDKIAKLKGLKDRLDKDIDSAQANVFVTNWDTARNQGALTYKDGSRFQLANAFMLAYDYGTPRLLSDYKWDVNDNGAPGATDTKVPDVDMGSACSANSSDWNCEQRWTSTRGMIAFHNYAGDAAVTDWQDDGDSNIAFSRDGKGFIAINNSKDDKNASYTTSLADGEYCDVYAVMDCSKTVTVKGGKVETKIPAHSAIALYAGATKTSHPQASAAKDPSDPDIGQQDDEVSATDKTVTIYYKPANATGRPPRSTTVWAMTGTSRKPT

SEQ ID NO: 18 - protein, 560 aa, Artificial Sequence
  maltotriose amylase variant
MMRKKSFWLGMLTAFMLVFTMAFSDSASAQPEARTSAKKDVQVIAFQQSWNTIAKECTDTYGPEGVGYVEVSPPQESIQGTQWWTSYQPVSYKLDSKLGTEAEFKNMIKQCNAAGVDIIADVVINHTTGSDVAAGDQKGVAGSDYNGSTGTYPGFATSQYPGGITDSDFHGCKRNISDYTNQQEVQECRLSSMWDFDTESEKVQDIQSDYLVDLWNAGVRAFRMDAVKHINTNSMKALKEKFAQKIGKNANDIYWIQEVIGNSSEASGIQPSNYLQNGTVTQFGFKSEMNQAFNDKIAKLKGLKDRLDKDIDSAQANVFVTNWDTARNQGALTYKDGSRFQLANAFMLAYDYGTPRLLSDYKWDVADNGAPGATDTKVPDVDMGSACSANSSDWNCEQRWTSTRGMIAFHNYAGDAAVTDWQDDGDSNIAFSRDGKGFIAINNSKDDKNASYTTSLADGEYCDVYAVMDCSKTVTVKGGKVETKIPAHSAIALYAGATKTSHPQASAAKDPSDPDIGQQDDEVSATDKTVTIYYKPANATGRPPKSTTVWAMTGTSRKPT

SEQ ID NO: 19 - protein, 560 aa, Artificial Sequence
  maltotriose amylase variant
MMRKKSFWLGMLTAFMLVFTMAFSDSASAQPEARTSAKKDVQVIAFQQNWNTIAKECTDTYGPEGVGYVEVSPPQESIQGTQWWTSYQPVSYKLDSKLGTEAEFKNMIKQCNAAGVDIIADVVINHTTGSDVAAGDQKGVAGSDYNGSTGTYPGFATSQYPGGITDSDFHGCKRNISDYTNQQEVQECRLSSMWDFDTESEKVQDIQSDYLVSLWNAGVRAFRMDAVKHINTNSMKALKEKFAQKIGKNANDIYWIQEVIGNSSEASGIQPSNYLQNGDVTQFGFKSEMNQAFKDKIAKLKGLKDRLDKDIDSAQANVFVTNWDTARNQGALTYKDGSRFQLANAFMLAYDYGTPRLLSDYKWDVNDNGAPGATDTKVPDVDMGSACSANSSDWNCEQRWTSTRGMIAFHNYAGDAAVTDWQDDGDSNIAFSRDGKGFIAINNSKDDKNASYTTSLADGEYCDVYAVMDCSKTVTVKGGKVETKIPAHSAIALYAGATKTSHPQASAAKDPSDPDIGQQDDEVSATDKTVTIYYKPANATGRPPRSTTVWAMTGTSRKPT

SEQ ID NO: 20 - protein, 560 aa, Artificial Sequence
  maltotriose amylase variant
MMRKKSFWLGMLTAFMLVFTMAFSDSASAQPEARTSAKKDVQVIAFQQSWNTIAKECTDTYGPEGVGYVEVSPPQESIQGTQWWTSYQPVSYKIDSKLGTEAEFKNMIKQCNAAGVDIIADVVINHTTGSDVAAGDQKGVAGSDYNGSTGTYPGFATSQYPGGITDSDFHGCKRNISDYTNQQEVQECRLSSMWDFDTESEKVQDIQSDYLVSLWNAGVRAFRMDAVKHINTNSMKALKEKFAQKIGKNANDIYWIQEVIGNASEASGIQPSNYLQNGTVTQFGFKSEMNQAFNDKIAKLKGLKDRLDKDIDSAQANVFVTNWDTARNQGALTYKDGSRFQLANAFMLAYDYGTPRLLSDYKWDVNDNGAPGATDTKVPDVDMGSACSANSSDWNCEQRWTSTRGMIAFHNYAGDAAVTDWQDDGDSNIAFSRDGKGFIAINNSKDDKNASYTTSLADGEYCDVYAVMDCSKTVTVKGGKVETKIPAHSAIALYAGATKTSHPQASAAKDPSDPDIGIQDDEVSATDKTVTIYYKPANATGRPPRSTTVWAMTGTSRKPT

SEQ ID NO: 21 - protein, 560 aa, Artificial Sequence
  maltotriose amylase variant
MMRKKSFWLGMLTAFMLVFTMAFSDSASAQPEARTSAKKDVQVIAFQQSWNTIAKECTDTYGPEGVGYVEVSPPQESIQGTQWWTSYQPVSYKLDSKLGTETEFKNMIKQCNAAGVDIIADVVINHTTGSDVAAGDQKGVAGSDYNGSTGTYPGFATSQYPGGITDSDFHGCKRNISDYTNQQEVQECRLSSMWDFDTESEKVQDIQSDYLVSLWNAGVRAFRMDAVKHINTNSMKALKEKFAQKIGKNANDIYWIQEVIGNSSEASGIQPSNYLQNGTVTQFGFKSEMNQAFNDKIAKLKGLKDRLDKDIDSAQANVFVTNWDTARNQGALTYKDGSRFQLANAFMLAYDYGTPRLLSDYKWDVNDNGAPGATDTKVPDVDMGSACSSNSSDWNCEQRWTSTRGMIAFHNYAGDAAVTDWQDDGDSNIAFSRDGKGFIAINNSKDDKNASYTTSLADGEYCDVYAVMDCSKTVTVKGGKVETKIPAHSAIALYAGATKTSHPQASAAKDPSDPDIGQQDDEVSATDKTVTIYYKPANATGRPPRSTTVWAMTGTSRKPT

SEQ ID NO: 22 - protein, 560 aa, Artificial Sequence
  maltotriose amylase variant
MMRKKSFWLGMLTAFMLVFTMAFSDAASAQPEARTSAKKDVQVIAFQQSWNTIAKECTDTYGPEGVGYVEVSPPQESIQGTQWWTSYQPVSYKLDSKLGTEAEFKNMVKQCNAAGVDIIADVVINHTTGADVAAGDQKGVAGSDYNGSTGTYPGFATSQYPGGITDSDFHGCKRNISDYTNQQEVQECRLSSMWDFDSESEKVQDIQSDYLVSLWNAGVRAFRMDAVKHINTNSMKALKEKFAQKIGKNANDIYWIQEVIGNSSEASGIQPSNYLQNGTVTQFGFKSEMNQAFNDKIAKLKGLKDRLDKDIDSAQANVFVTNWDTARNQGALTYKDGSRFQLANAFMLAYDYGTPRLLSDYKWDVNDNGAPGATDTKVPDVDMGSACSANSSDWNCEQRWTSTRGMIAFHNYAGDAAVTDWQDDGDSNIAFSRDGKGFIAINNSKDDKNASYTTSLADGEYCDVYAVMDCSKTVTVKGGKVETKIPAHSAIALYAGATKTSHPQASAAKDPSDPDIGQQDDEVSATDKTVTIYYKPANATGRPPRSTTVWAMTGTSRKPT

SEQ ID NO: 23 - protein, 560 aa, Artificial Sequence
  maltotriose amylase variant
MMRKKSFWLGMLTAFMLVFTMAFSDSASAQPEARTSAKKDVQVIAFQQSWNTIAKECTDTYGPAGVGYVEVSPPQESIQGTQWWTSYQPVSYKLASKLGTEAEFKNMVKQCNAAGVDIIADVVINHTTGSDVAAGDQKGVAGSDYNGSTGTYPGFATSQYPGGITDSDFHDCKRNISDYTNQQEVQECRLSSMWDFDTESEKVQDIQSDYLVSLWNAGVRAFRMDAVKHINTNSMKALKEKFAQKIGKNANDIYWIQEVIGNASEASGIQPSNYLQNGTVTQFGFKSEMNQAFNDKIAKLKGLKDRLDKDIDSAQANVFVTNWDTARNQGALTYKDGSKFQLANAFMLAYDYGTPRLLSDYKWDVNDNGAPGATDTKVPDVDMGSACSANSSDWNCEQRWTSTRGMIAFHNYAGDAAVTDWQDDGDSNIAFSRDGKGFIAINNSKDDKNASYTTSLADGEYCDVYAVMDCSKTVTVKGGKVETKIPAHSAIALYAGATKTSHPQASAAKDPSDPDIGQQDDEVSATDKTVTIYYKPANATGRPPKSTTVWAMTGTSRKPT

SEQ ID NO: 24 - protein, 560 aa, Artificial Sequence
  maltotriose amylase variant
MMRKKSFWLGMLTAFMLVFTMAFSDVASAQPEARTSAKKDVQVIAFQQSWNTIAKECTDTYGPEGVGYVEVSPPQESIQGTQWWTSYQPVSYKLASKLGTEAEFKNMVKQCNAAGVDIIADVVINHTTGSDVAAGDQKGVAGSDYNGSTGTYPGFATSQYPGGITDSDFHDCKRNISDYTNQQEVQECRLSSMWDFDTESEKVQDIQSDYLVSLWNAGVRAFRMDAVKHINTNSMKALKEKFAQKIGKNANDIYWIQEVIGNASEASGIQPSNYLQNGTVTQFGFKSEMNQAFNDKIAKLKGLKDRLDKDIDSAQANVFVTNWDTARNQGALTYKDGSRFQLANAFMLAYDYGTPRLLSDYKWDVNDNGAPGATDTKVPDVDMGSACSANSSDWNCEQRWTSTRGMIAFHNYAGDAAVTDWQDDGDSNIAFSRDGKGFIAINNSKDDKNASYTTSLADGEYCDVYAVMDCSKTVTVKGGKVETKIPAHSAIALYAGATKTSHPQASAAKDPSDPDIGQQDDEVSATDKTVTIYYKPADATGRPPRSTTVWAMTGTSRKPT

SEQ ID NO: 25 - protein, 560 aa, Artificial Sequence
  maltotriose amylase variant
MMRKKSFWLGMLTAFMLVFTMAFSDAASAQPEARTSAKKDVQVIAFQQSWNTIAKECTDTYGPEGVGYVEVSPPQESIQGTQWWTSYQPVSYKLASKLGTEAEFKNMVKQCNAAGVDIIADVVINHTTGSDVAAGDQKGVAGSDYNGSTGTYPGFATSQYPGGITDSDFHDCKRNISDYTNQQEVQECRLSSMWDFDTESEKVQDIQSDYLVSLWNAGVRAFRMDAVKHINTNSMKALKEKFAQKIGKNANDIYWIQEVIGNSSEASGIQPSNYLQNGTVTQFGFKSEMNQAFNDKIAKLKGLKDRLDKDIDSANANVFVTNWDTARNQGALTYKDGSRFQLANAFMLAYDYGTPRLLSDYKWDVNDNGAPGATDTKVPDVDMGSACSTNSSDWNCEQRWTSTRGMIAFHNYAGDAAVTDWQDDGDSNIAFSRDGKGFIAINNSKDDKNASYTTSLADGEYCDVYAVMDCSKTVTVKGGKVETKIPAHSAIALYAGATKTSHPQASAAKDPSDPDIGQQDDEVSATDKTVTIYYKPANATGRPPRSTTVWAMTGTSRKPT

SEQ ID NO: 26 - protein, 560 aa, Artificial Sequence
  maltotriose amylase variant
MMRKKSFWLGMLTAFMLVFTMAFSDVASAQPEARTSAKKDVQVIAFQQSWNTIAKECTDTYGPEGVGYVEVSPPQESIQGTQWWTSYQPVSYKLASKLGTEAEFKNMVKQCNAAGVDIIADVVINHTTGSDVAAGDQKGVAGSDYNGSTGTYPGFATSQYPGGITDSDFHDCKRNISDYTNQQEVQECRLSSMWDFDTESEKVQDIQSDYLVSLWNAGVRAFRMDAVKHINTNSMKALKEKFAQKIGKNANDIYWIQEVIGNASEASGIQPSNYLQNGTVTQFGFKSEMNQAFNDKIAKLKGLKDRLDKDIDSAQANVFVTNWDTARNQGALTYKDGSRFQLANAFMLAYDYGTPRLLSDYKWDVNDNGAPGATDTKVPDVDMGSACSANSSDWNCEHRWTSTRGMVAFHNYAGDAAVTDWQDDGDSNIAFSRDAKGFIAINNSKDDKNASYTTSLADGEYCDVYAVMDCSKTVTVKGGKVETKIPAHSAIALYAGATKTSHPQASAAKDPSDPDIGQQDDEVSATDKTVTIYYKPADATGRPPRSTTVWAMTGTSRKPT

SEQ ID NO: 27 - protein, 560 aa, Artificial Sequence
  maltotriose amylase variant
MMRKKSFWLGMLTAFMLVFTMAFSDVASAQPEARTSAKKDVQVIAFQQSWNTIAKECTDTYGPEGVGYVEVSPPQESIQGTQWWTSYQPVSYKLASKLGTEAEFKNMVKQCNAAGVDIIADVVINHTTGSDVAAGDQKGVAGSDYNGSTGTYPGFATSQYPGGITDSDFHDCKRNISDYTNQQEVQECRLSSMWDFDTESEKVQDIQSDYLVSLWNAGVRAFRMDAVKHINTNSMKALKEKFAQKIGKNANDIYWIQEVIGNASEASGIQPSNYLQNGTVTQFGFKSEMNQAFNDKIAKLKGLKDRLDKDIDSAQANVFVTNWDTARNQGALTYKDGSRFQLANAFMLAYDYGTPRLLSDYKWDVNDNGAPGATDTKVPDVDMGSACSANSSDWNCEQRWTSTRGMIAFHNYAGDAALTDWQDDGDNNIAFSRDGKGFIAINNSKDDKNASYTTSLADGEYCDVYAVMDCSQTVTVKGGKVETKIPAHSAIALYAGATKTSHPQASAAKDPSDPDIGQQDDEVSATDKTVTIYYKPADATGRPPRSTTVWAMTGTSRKPT

SEQ ID NO: 28 - protein, 560 aa, Artificial Sequence
  maltotriose amylase variant
MMRKKSFWLGMLTAFMLVFTMAFSDVASAQPEARTSAKKDVQVIAFQQSWNTIAKECTDTYGPEGVGYVEVSPPQESIQGTQWWTSYQPVSYKLASKLGTEAEFKNMVKQCNAAGVDIIADVVINHTTGSDVAAGDQKGVAGSDYNGSTGTYPGFATSQYPGGITDSDFHDCKRNISDYTNQQEVQECRLSSMWDFDTESEKVQDIQSDYLVSLWNAGVRAFRMDAVKHINTNSMKALKEKFAQKIGKNANDIYWIQEVIGNASEASGIQPSNYLQNGTVTQFGFKSEMNQAFNDKIAKLKGLKDRLDKDIDSAQANVFVTNWDTARNQGALTYKDGSRFQLANAFMLAYDYGTPRLLSDYKWDVNDNGAPGATDTKVPDVDMGSACSANSSDWNCEQRWTSTRGMVAFHNYAGDAAVTDWQDDGDNNIAFSRDGKGFIAINNSKDDKNASYTTSLADGEYCDVYAVMDCSKTVTVKGGKVEAKIPAHSAIALYAGATKTSHPQASAAKDPSDPDIGQQDDEVSATDKTVTIYYKPADATGRPPRSTTVWAMTGTSRKPT

SEQ ID NO: 29 - protein, 560 aa, Artificial Sequence
  maltotriose amylase variant
MMRKKSFWLGMLTAFMLVFTMAFSDVASAQPEARTSAKKDVQVIAFQQSWNTIAKECTDTYGPEGVGYVEVSPPQESIQGTQWWTSYQPVSYKLASKLGTEAEFKNMVKQCNAAGVDIIADVVINHTTGSDVAAGDQKGVAGSDYNGSTGTYPGFATSQYPGGITDSDFHDCKRNISDYTNQQEVQECRLSSMWDFDTESEKVQDIQSDYLVSLWNAGVRAFRMDAVKHINTNSMKALKEKFAQKIGKNANDIYWIQEVIGNASEASGIQPSNYLQNGTVTQFGFKSEMNQAFNDKIAKLKGLKDRLDKDIDSAQANVFVTNWDTARNQGALTYKDGSRFQLANAFMLAYDYGTPRLLSDYKWDVNDNGAPGATDTKVPDVDMGSACSANSSDWNCEQRWTSTRGMIAFHNYAGDAAVTDWQDDGDNNIAFSRDAKGFIAINNSKDDKNASYTTSLADGEYCDVYAVMDCSKAVTVKGGKVETKIPAHSAIALYAGATKTSHPQASAAKDPSDPDIGQQDDEVSATDKTVTIYYKPADATGRPPRSTTVWAMTGTSRKPT

SEQ ID NO: 30 - protein, 29 aa, Bacillus licheniformis
MMRKKSFWLGMLTAFMLVFTMAFSDSASA

SEQ ID NO: 31 - protein, 29 aa, Artificial Sequence
  variant of Bacillus licheniformis keratinase signal peptide
MMRKKSFWLGMLTAFMLVFTMAFSDAASA

SEQ ID NO: 32 - protein, 29 aa, Artificial Sequence
  variant of Bacillus licheniformis keratinase signal peptide
MMRKKSFWLGMLTAFMLVFTMAFSDVASA

(19) United States
(12) Patent Application Publication
(10) Pub. No.: US 2026/0258384 A1
(43) Pub. Date: Sep. 3, 2026

(54) THERMOSTABLE MALTOTRIOSE AMYLASE PROTEIN

(71) Applicant: Archer Daniels Midland Company, Decatur, IL (US)

(72) Inventors: Padmesh Venkitasubramanian, Forsyth, IL (US); Ashley Garner, Sacramento, CA (US); S M Mahfuzul Islam, Decatur, IL (US); Bishnu Subedi, Forsyth, IL (US); Mark Welch, Fremont, CA (US); Sridhar Govindarajan, Los Altos, CA (US)

(21) Appl. No.: 18/264,836

(22) PCT Filed: Feb. 7, 2022
(86) PCT No.: PCT/US2022/015514

§ 371 Date: Aug. 9, 2023

Related U.S. Application Data
(60) Provisional application No. 63/147,472, filed on Feb. 9, 2021.

(51) Int. Cl. C12N 9/28; C12P 19/02; C12P 19/14; C13K 1/06

(52) U.S. Cl. CPC C12N 9/2417; C12P 19/02; C12P 19/14; C12Y 302/01001; C13K 1/06

(57) ABSTRACT

This disclosure provides maltotriose amylase enzymes useful for commercial scale production of reduced sugar glucose syrups (RSGS) from a starch. Useful enzymes (“amylase variants” or “enzyme variants”) may be variants of a Bacillus subtilis amylase or enzymes with at least 90% sequence identity thereto engineered with mutations that improve thermal tolerance and activity at a temperature above 50° C. as compared with the parent enzymes.

### CROSS-REFERENCE TO RELATED APPLICATIONS

This application is a national stage entry of International Application No. PCT/US22/015514, filed Feb. 7, 2022, which itself claims priority to U.S. Provisional Patent Application No. 63/147,472, filed Feb. 9, 2021, each of the contents of the entirety of which are incorporated by this reference.

### SEQUENCE SUBMISSION

The instant application contains a sequence listing which has been submitted in ASCII format and is hereby incorporated by reference in its entirety. Said ASCII copy, created on Dec. 17, 2025, is named “ADM CP 0201 US00 substitute_ST25.txt” and is 143,402 bytes in size.

### BACKGROUND OF THE INVENTION

In 2015, the Dietary Guidelines Advisory Committee of the United States Department of Agricultural recommended to limit added sugar intake to less than 10% of total daily calories. In May 2016, the Food and Drug Administration issued new labeling guidelines to align with these recommendations. An “added sugars” term was added to the nutritional facts panel for labeling of foods and the Committee defined added sugars as being mono and disaccharides. The new added sugar regulations went into effect on Jul. 26, 2018. “Added sugars,” in grams and as percent Daily Value, was required to be included on food labels. Scientific data shows that it is difficult to meet nutrient needs while staying within calorie limits if a person consumes more than 10 percent of their total daily calories from added sugar, and this is consistent with the 2015-2020 Dietary Guidelines for Americans.

To address this change in FDA regulation, methods have been sought to make reduced sugar glucose syrups that allows food manufacturers to reduce the amount of “total sugar” and “added sugars” on labels for foods containing these reduced sugar syrups.

Current methods to make functionally well-received reduced sugar glucose syrups (RSGS) have drawbacks and limitations. Conventional methods involve using commodity enzymes that are do not to produce a desired oligosaccharide profile for a RSGS. The oligosaccharide profile has an impact on the finished product viscosity, sweetness and water activity. The levels of DP10+ oligosaccharide will have an impact on finished syrup viscosity which in turn affects end use applications for the syrup in foods. The enzymes currently used to achieve desired oligosaccharide profiles for a RSGS are costly specialty enzymes that impact the cost of providing the finished RSGS product and therefore is a stumbling block for market adaptation.

There is a need for methods that do not have the drawbacks of the current methods while overcoming the limitations of conventional methods. In particular, there is a need for thermostable enzymes for making reduced sugar syrups that have a least one improved characteristic over existing commodity or specialty enzymes. For example, there is a need for thermal stable enzymes that have optimal activity at temperatures greater than 50° C., which is the typical temperatures of conventional process. In this regard there is a need for thermostable enzymes that can operate at temperatures of greater than 50° C. There is also a need for source of thermostable enzymes that allows for improved enzyme production by fermentation and downstream processing.

### BRIEF SUMMARY

The present invention provides advantages over commodity/specialty enzymes used for the production of low sugar syrups. In one aspect, provided herein are family of maltotriose amylase enzymes having improved properties that comprise a variant maltotriose amylase that contains an amino acid sequence that includes the mutations present in a variant maltotriose amylase selected from the group consisting of amino acids 30-560 of SEQ ID NOS: 2-29.

In most embodiments, the variant maltotriose amylase is a variant of the maltotriose enzyme according SEQ ID NO:1, wherein the variant comprises a Q126H amino acid substitution.

In exemplary embodiments, the variant maltotriose enzyme comprises the mutations present in the variant maltotriose enzymes selected from the group consisting of the maltotriose enzymes according to amino acids 30-560 of SEQ ID NOS: 2-29. In particular embodiment the variant maltotriose enzyme has an amino acid sequence according any of SEQ ID NOS: 2-29. In certain embodiments the variant maltotriose amylase is expressed from Bacillus subtilis. In such embodiments, the variant maltotriose amylase includes a signal peptide for export of the enzyme from Bacillus subtilis. The signal peptide may be one with an amino acid sequence selected from the group consisting of the SEQ ID NO:30-32.

In another aspect, methods are provided that comprise contacting starch with any of the forgoing maltotriose amylase enzymes at a temperature of above 50° C. to form a reduced sugar glucose syrup (RSGS). In a preferred embodiment, the RSGS has a dextrose equivalent (DE) of 25-40.

The present invention provides improved processing techniques over conventional methods. The present disclosure provides thermostable enzymes that can operate at temperatures of 60-80° C. In particular embodiments, the present disclosure provides for forming a RSGS using thermostable maltotriose amylase enzymes that operate at temperatures of 60-80° C., with an increase in catalytic activity.

These and other aspects, embodiments, and associated advantages will become apparent from the following Detailed Description.

### BRIEF DESCRIPTION OF THE DRAWINGS

FIG. 1 is a bar chart showing the fraction of total saccharides present in reduced sugar glucose syrups produced by various natural maltotriose amylase enzymes and methods disclosed herein in terms of distribution by degree of polymerization (DP) obtained by digestion of Clintose CR10 an exemplary maltodextrin solution) present at a 30% (w/w) dissolved solids concentration.

FIG. 2 is a graph showing improvement in thermal stability and proteolytic stability in whole broth after round 3 of protein engineering using exemplary variant maltotriose amylase enzymes s according to the present disclosure.

FIG. 3 is a bar chart showing comparative stability of exemplary variant maltotriose amylase enzymes (as characterized by half-life) in whole broth when heated at 57° C. and 55° C.

FIG. 4 is a bar chart showing hydrolysis of maltodextrin (Clintose CR10) by enzyme variants at 56° C.

FIG. 5 is a bar chart showing hydrolysis of maltodextrin (Clintose CR10) by enzyme variants at 63° C.

### DETAILED DESCRIPTION

Thermal stable maltotriose amylase enzymes have now been discovered that retain enzymatic activity above 50° C. and in some embodiments up to 70° C. Enzymes disclosed herein have the following characteristics: (a) hydrolyzes hydrolyzed Starch or maltodextrin to primarily DP3 with minimal DP1 and DP2 content; (b) are active with substrate present at 30% dissolved solids (d.s.) maltodextrin, pH 5-6, at 60-65° C.; (c) are stable at 70° C. for greater than 30 minutes; (d) are inactivated at pH 3.5; and (e) is expressed well in Bacillus subtilis.

The enzymes disclosed herein can be used to make a reduced sugar glucose syrup (RSGS) having a high concentration of DP3, i.e., greater than 40%, while making a low concentration of DP1 and DP2, i.e., DP1+DP2 less than 10%. In exemplary embodiments, the ratio of DP3 to DP2 in the syrup is at least 6. In a preferred embodiment, the RSGS has a dextrose equivalent (DE) of 24-40.

### Enzyme Development

A derivative of Bacillus subtilis strain DP1077 was created wherein the native amyE gene was disrupted. This strain was designated DP1384, which was used as the host to express variant maltotriose amylase enzymes (a.k.a., DP3 amylase). To ensure that the observed activity in the culture supernatant was from the expressed DP3 amylase and not from endogenous amylase, two known DP3 amylases were expressed in strain DP1384 and the strains were cultured. The maltotriose amylase natural homologs were expressed and secreted using one of three signal peptides on of which is the Bacillus licheniformis keratinase signal peptide (MMRKKSFWLGMLTAFMLVFTMAFSDAASA (SEQ ID NO;30); amino acids 1-29 of SEQ ID NO:1). The other two signal peptides were slight variants of the forgoing, which were MMRKKSFWLGMLTAFMLVFTMAFSDAASA (SEQ ID NO: 31) and MMRKKSFWLGMLTAFMLVFTMAFSDVASA (SEQ ID NO: 32) where the underlining shows the amino acid variation. The culture supernatants were tested for enzyme activity using 3,5-dinitrosalicyclic acid (DNS) as an indicator.

It was found that amylase activity was present in the culture supernatant of the wild type strain DP1077 and in cultures where DP3 amylase was overexpressed, but not in host strain DP1384 where the amylase gene amyE was knocked out. This proved that the observed activity in the culture supernatant was from the expressed DP3 amylase and not from any endogenous amylase expressed in the host strain DP1077.

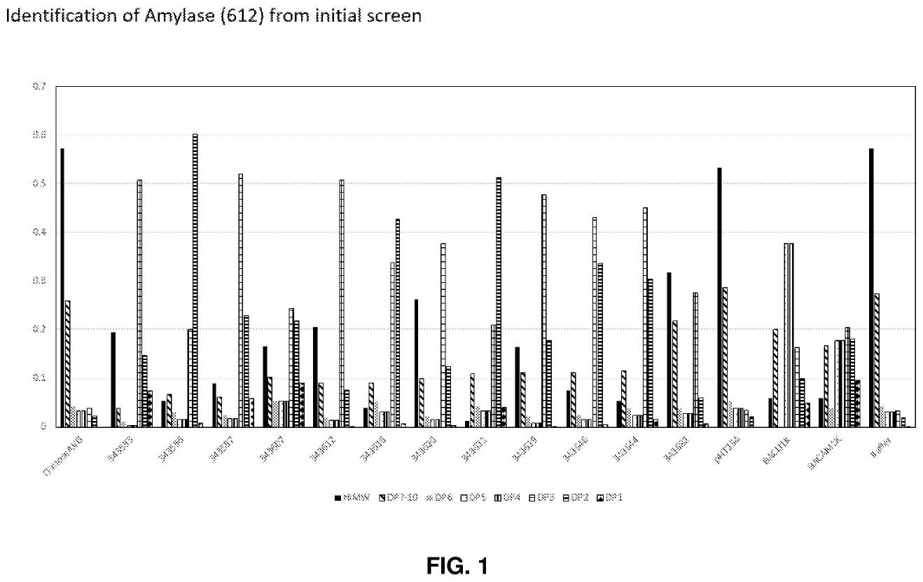

Genes for ninety-six (96) natural amylase homologs were codon optimized, synthesized, cloned and expressed in B. subtilis DP1384. The strains expressing DP3 amylases were cultured in LB media (i.e., lysogeny broth, also referred to as Luria-Bertani broth) with antibiotics. The culture supernatant was assayed for protein expression and for amylase activity by DNS using Clintose CR10 as a substrate. Clintose CR10 is a tradename for a maltodextrin produced by the Archer Daniels Midland Company characterized as having a dextrose equivalent (DE value) of 10. Reactions were carried out in the presence of 8% w/w Clintose CR10 (d.s.), and the reaction product was characterized by HPLC to determine the DP profile. Homolog enzymes that gave desired desirable DP profile were further characterized by performing the reaction in the presence of 30% (w/w) Clintose CR10 to understand reaction rate and product spectrum. The results are shown in FIG. 1.

Based on the analysis, the maltotriose amylase gene designated 343612 (612) having the amino acid sequence according to SEQ ID: NO: 1 was taken as lead candidate for protein engineering. 343612 had better expression compared to other candidates, gave a lower DP2+1 concentration (<10%) and was catalytically active at 50° C. for starch hydrolysis.

Protein engineering was used to improve the properties of the 343612 amylase. Protein engineering entailed synthesizing genes encoding variants of SEQ ID: NO: 1 containing random mutations in various regions of the parent 343612 gene sequence to alter the amino acid sequence followed by screening for mutations that had improved properties. The initial round of mutation was followed by further rounds of mutation retaining mutations from the first round variants that had improved properties to discover mutations that further improved properties when stacked with favorable mutations from the first round.

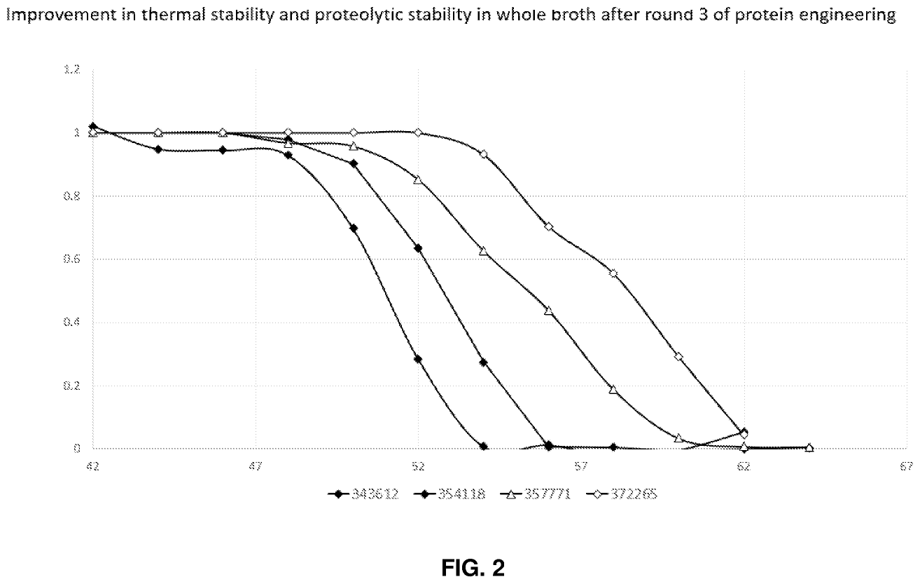

Improvement in thermal stability and proteolytic stability in whole broth was achieved after round 3 of protein engineering. The results are shown in FIG. 2. FIG. 2 t shows the temperature versus activity properties determined using 343612 (R1 parent), 354118 (R2 parent), 357771 (R3 parent), and 372265 (R4 parent). 354118 (R2 parent) did not outperform 343612 (R1 parent) at any pre-treat temperature. 357771 (R3 parent) and 372265 (R4 parent), however, outperformed 343612 (R1 parent) at pre-treat temperatures above 50° C. In the forgoing sentences RX parent refers to the parent sequence for a given round (R) of mutation designated by X.

### Alternative Variant Enzymes

### Corn Syrup Process Development with Maltotriose Amylase

In an aspect, a method comprises contacting starch with a thermostable maltotriose amylase enzyme comprising an amino acid sequence having the mutations present in the variant enzymes selected from the group consisting of amino acids 30-560 of SEQ ID NOS: 2-29 at a temperature of above 50° C. to form a reduced sugar glucose syrup with a dextrose equivalent (DE) of 24-40. In a preferred embodiment, the reduced sugar glucose syrup (RSGS) has a viscosity similar to DE 42/43 syrup of ~7000 cps, higher DP3 and DP3+DP4 and lower DP1+2 than DE 42/43 syrup

In one preferred embodiment, the variant thermostable maltotriose amylase protein comprises an amino acid sequence selected from the group consisting of amino acid sequences 30-560 of SEQ ID NO:1. In another preferred embodiment the thermostable maltotriose amylase protein comprises an amino acid sequence selected from the group consisting of amino acid sequences 30-560 of SEQ ID NO:1. but for having at least one substitution being Q126H. Amylase variants (also referred herein as “variant enzymes” or “variant maltotriose amylase”) having at least the Q126H substitution were found to have increased thermostability above 50° C. when compared to the parent maltotriose amylase.

In a preferred embodiment, a variant thermostable maltotriose amylase protein has at least one improved characteristic when compared to the parent maltotriose amylase, wherein the improved characteristic is selected from the group consisting of increased thermostability above 50° C., better activity in the presence of a substrate maltodextrin at 30% wt/wt dissolved solids content, and better protein expression of the thermostable maltotriose amylase when a gene encoding the thermostable maltotriose amylase is expressed in a Bacillus subtilis host cell.

Four (4) rounds of genetic modification and screening were performed, and five (5) high performing variants were selected. Variants were also screened for activity at pH 5.5 and 6. Temperature was increased to range between 60° C.-69° C.

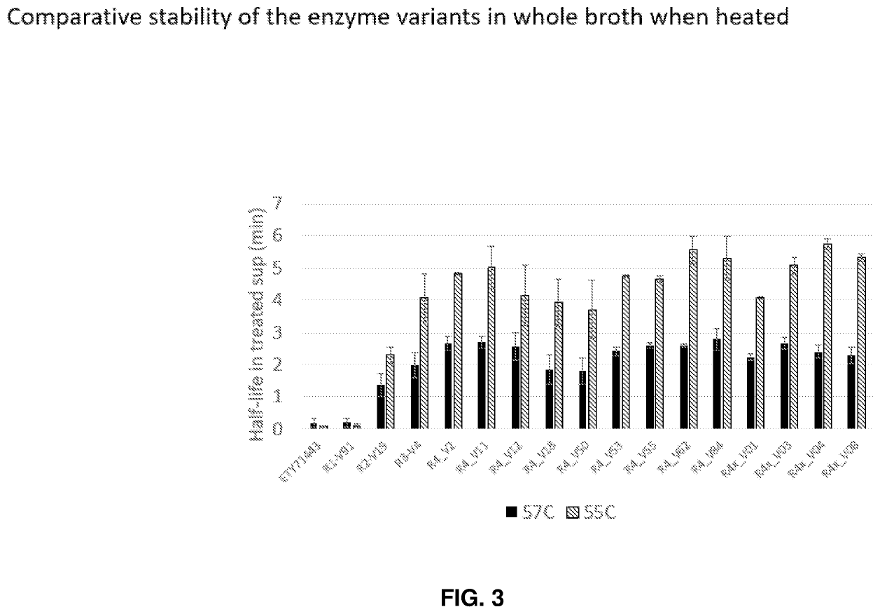

FIG. 3 is a bar chart showing comparative stability of the enzyme variants (as characterized by half-life) in assayed using the supernatants from lysed cultures expressing the variants and assayed. at 57° C. and 55° C.

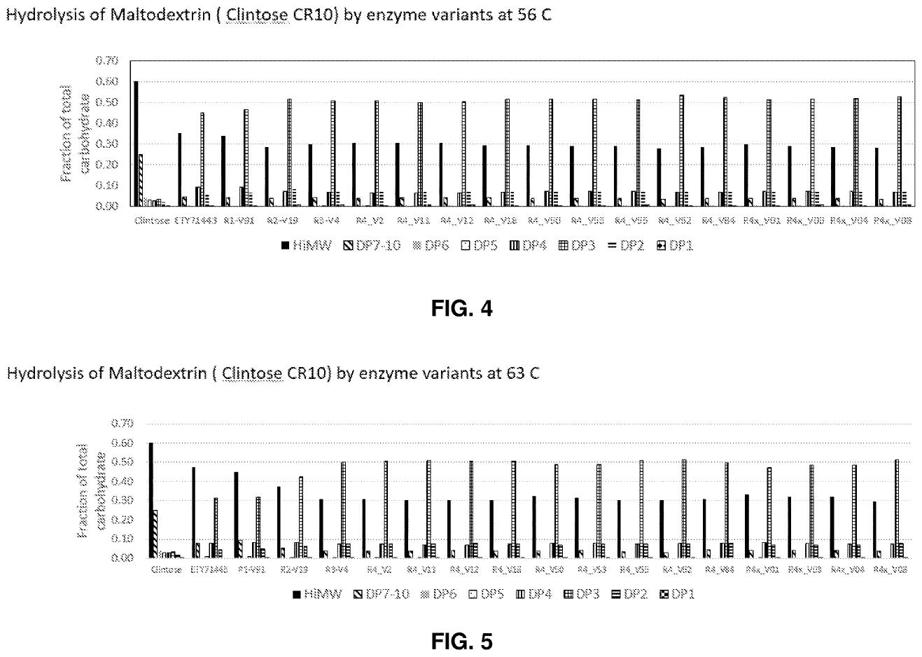

FIG. 4 is a bar chart showing hydrolysis of maltodextrin (Clintose CR10) by enzyme variants at 56° C.

FIG. 5 is a bar chart showing hydrolysis of maltodextrin (Clintose CR10) by enzyme variants at 63° C.

Table 1 shows amino acid changes of exemplary variants according the present invention relative to the amino acid sequence of SEQ ID NO:1.

All amino acid substitution mutations described herein are presented in the form XNY, where X is the one letter code for the amino acid at position N and Y is the substitute amino acid at that position. In the event of a discrepancy between the amino acid substitutions in the tables herein and the sequences provided in the sequence listing, the amino acid substitutions shown in the tables control.

Table 2 summarizes the amino acid substitutions relative to SEQ ID NO:1 and sequence identifiers of certain variants.

Those having skill in the art, with the knowledge gained from the present disclosure, will recognize that various changes can be made to the disclosed processes in attaining these and other advantages, without departing from the scope of the present disclosure. As such, it should be understood that the features of the disclosure are susceptible to modifications and/or substitutions. The specific embodiments illustrated and described herein are for illustrative purposes only, and not limiting of the invention as set forth in the appended claims.

TABLE 1
Maltotriose Amylase Variants

| GeneID | Amino Acid Substitutions Relative to SEQ ID NO: 1 | | | | | | | | | | | | | |
|---|---|---|---|---|---|---|---|---|---|---|---|---|---|---|
| 354028 | K38A | A67G | I253A | | | | | | | | | | | |
| 354029 | T128A | Q137G | I253A | | | | | | | | | | | |
| 354030 | N175D | I253A | G372N | | | | | | | | | | | |
| 354031 | A45L | D209A | G372N | | | | | | | | | | | |
| 354032 | A102T | I206A | T333N | | | | | | | | | | | |
| 354033 | S177N | S234D | A389S | | | | | | | | | | | |
| 354034 | N181D | I297R | N366R | | | | | | | | | | | |
| 354035 | T165Q | I176V | T333S | | | | | | | | | | | |
| 354036 | D25A | S208A | V365N | | | | | | | | | | | |
| 354037 | R174A | N315K | G372N | | | | | | | | | | | |
| 354038 | S49N | K93R | M193L | | | | | | | | | | | |
| 354039 | S177N | A221G | Y350W | | | | | | | | | | | |
| 354040 | A45L | S49N | K294N | | | | | | | | | | | |
| 354041 | Q126H | G372N | S385N | | | | | | | | | | | |
| 354042 | Q126H | T333N | N390D | | | | | | | | | | | |
| 354043 | S49R | A102T | I297R | | | | | | | | | | | |
| 354044 | S26A | I206A | N366A | | | | | | | | | | | |
| 354045 | S49R | E103A | F196L | | | | | | | | | | | |
| 354046 | S49N | F285Y | A389S | | | | | | | | | | | |
| 354047 | K93R | S167A | I253A | | | | | | | | | | | |
| 354048 | D59N | T149D | N175D | | | | | | | | | | | |
| 354049 | S26A | I176V | I253A | | | | | | | | | | | |
| 354050 | Q137G | I176V | D252N | | | | | | | | | | | |
| 354051 | Q137G | S198T | T333S | | | | | | | | | | | |
| 354052 | T149D | S177N | S272G | | | | | | | | | | | |
| 354053 | D59N | T165Q | S234D | | | | | | | | | | | |
| 354054 | D25A | R174A | S272G | | | | | | | | | | | |
| 354055 | S49N | I206A | N366R | | | | | | | | | | | |
| 354056 | Q137G | T149D | N366A | | | | | | | | | | | |
| 354057 | A120V | V122A | S385N | | | | | | | | | | | |
| 354058 | T149D | N315Q | T333S | | | | | | | | | | | |
| 354059 | T165Q | K294N | N315K | | | | | | | | | | | |
| 354060 | N51A | K93R | F285Y | | | | | | | | | | | |
| 354061 | A45L | N51A | S263A | | | | | | | | | | | |
| 354062 | N233A | S263A | Y350W | | | | | | | | | | | |
| 354063 | E103A | N366R | A389S | | | | | | | | | | | |
| 354064 | F196L | S198T | V365N | | | | | | | | | | | |
| 354065 | A45L | I297R | A389S | | | | | | | | | | | |
| 354066 | A120V | N181D | S208A | | | | | | | | | | | |
| 354067 | D25A | N51A | N366A | | | | | | | | | | | |
| 354068 | A45L | I176V | W363F | | | | | | | | | | | |
| 354069 | A67G | M193L | F285Y | | | | | | | | | | | |
| 354070 | A102T | S234D | S272G | | | | | | | | | | | |
| 354071 | S28A | D59N | I297R | | | | | | | | | | | |
| 354072 | D252N | S385N | A389S | | | | | | | | | | | |
| 354073 | D25A | S28A | V122A | | | | | | | | | | | |
| 354074 | T232A | S263A | N390D | | | | | | | | | | | |
| 354075 | D59N | R174A | D209A | | | | | | | | | | | |
| 354076 | S28A | I206A | N233A | | | | | | | | | | | |
| 354077 | M193L | Y350W | V365N | | | | | | | | | | | |
| 354078 | T128A | S177N | S198T | | | | | | | | | | | |
| 354079 | S28A | S167A | N175D | | | | | | | | | | | |
| 354080 | A102T | A237N | S385N | | | | | | | | | | | |
| 354081 | N175D | T333S | Y350W | | | | | | | | | | | |
| 354082 | N181D | D209A | N315K | | | | | | | | | | | |
| 354083 | R174A | I206A | S263A | | | | | | | | | | | |
| 354084 | T128A | F196L | A221G | | | | | | | | | | | |
| 354085 | A67G | A102T | N233A | | | | | | | | | | | |
| 354086 | N175D | A221G | A237N | | | | | | | | | | | |
| 354087 | S263A | S272G | N366A | | | | | | | | | | | |
| 354088 | S26A | N181D | T232A | | | | | | | | | | | |
| 354089 | K93R | S208A | T333N | | | | | | | | | | | |
| 354090 | A120V | S198T | W363F | | | | | | | | | | | |
| 354091 | N181D | A221G | T333N | | | | | | | | | | | |
| 354092 | M193L | N315K | N366A | | | | | | | | | | | |
| 354093 | K38A | S167A | W363F | | | | | | | | | | | |
| 354094 | A67G | E103A | K294N | | | | | | | | | | | |
| 354095 | S177N | A237N | N315Q | | | | | | | | | | | |
| 354096 | E103A | V122A | S208A | | | | | | | | | | | |
| 354097 | E103A | A221G | S385N | | | | | | | | | | | |
| 354098 | S26A | K38A | T333N | | | | | | | | | | | |
| 354099 | V122A | I176V | F196L | | | | | | | | | | | |
| 354100 | S49R | Q126H | W363F | | | | | | | | | | | |
| 354101 | Q126H | S198T | T232A | | | | | | | | | | | |
| 354102 | N51A | F196L | D252N | | | | | | | | | | | |
| 354103 | T128A | T232A | N233A | | | | | | | | | | | |
| 354104 | K38A | A120V | D252N | | | | | | | | | | | |
| 354105 | D209A | F285Y | T333S | | | | | | | | | | | |
| 354106 | D59N | N233A | N390D | | | | | | | | | | | |
| 354107 | K93R | N315Q | V365N | | | | | | | | | | | |
| 354108 | K38A | S234D | F285Y | | | | | | | | | | | |
| 354109 | T128A | D252N | Y350W | | | | | | | | | | | |
| 354110 | S49R | T232A | N315K | | | | | | | | | | | |
| 354111 | S49R | M193L | N366R | | | | | | | | | | | |
| 354112 | K294N | N366R | G372N | | | | | | | | | | | |
| 354113 | D25A | D209A | N315Q | | | | | | | | | | | |
| 354114 | T165Q | S208A | I297R | | | | | | | | | | | |
| 354115 | T149D | A237N | N390D | | | | | | | | | | | |
| 354116 | V122A | Q137G | R174A | | | | | | | | | | | |
| 354117 | S28A | S272G | W363F | | | | | | | | | | | |
| 354118 | K294N | N315Q | N390D | | | | | | | | | | | |
| 354119 | T165Q | S167A | V365N | | | | | | | | | | | |
| 354120 | A120V | Q126H | S167A | | | | | | | | | | | |
| 354121 | A67G | S234D | A237N | | | | | | | | | | | |
| 354122 | S26A | S49N | N51A | | | | | | | | | | | |
| 357753 | A67G | N233A | S272G | K294N | N315Q | N390D | | | | | | | | |
| 357754 | A102T | E103Q | T232A | K294N | N315Q | N390D | | | | | | | | |
| 357755 | W50F | K294N | N315Q | V365N | N390D | A507T | | | | | | | | |
| 357756 | W50F | K294N | N366A | N390D | | | | | | | | | | |
| 357757 | K55R | K286A | K294N | N315Q | V365N | N390D | | | | | | | | |
| 357758 | A221G | A250R | N315Q | N390D | | | | | | | | | | |
| 357759 | S26A | N233A | K294R | N315Q | N390D | | | | | | | | | |
| 357760 | A102T | S177A | K294R | N315Q | N390D | | | | | | | | | |
| 357761 | I118V | A221G | K286A | K294N | N315Q | N390D | | | | | | | | |
| 357762 | S167A | K294N | N315Q | T333S | N366A | N390D | | | | | | | | |
| 357763 | S49N | G171D | K294N | N315Q | S385N | N390D | | | | | | | | |
| 357764 | S49N | K294N | D360G | N390D | | | | | | | | | | |
| 357765 | K286A | K294N | N315Q | T333S | N390D | A525P | | | | | | | | |
| 357766 | S177N | K294N | N315Q | V365N | S385N | N390D | | | | | | | | |
| 357767 | A67G | K294N | N315Q | N390D | A507T | | | | | | | | | |
| 357768 | A102T | T127M | S177N | K294N | N315Q | N390D | | | | | | | | |
| 357769 | S49N | E103Q | S177N | K294N | N315Q | N390D | | | | | | | | |
| 357770 | E187H | N233A | K294N | N315Q | N390D | A507T | | | | | | | | |
| 357771 | A67G | Q126H | K294N | N315Q | | | | | | | | | | |
| 357772 | T127M | S167A | K294N | N315Q | N390D | Q518I | | | | | | | | |
| 357773 | E201D | K294N | N390D | | | | | | | | | | | |
| 357774 | N51A | A120V | G171D | K294N | N315Q | N390D | | | | | | | | |
| 357775 | Q126H | K294N | D310A | N315Q | A389S | N390D | | | | | | | | |
| 357776 | S177N | S272G | K294N | N315Q | R339K | N390D | | | | | | | | |
| 357777 | A67G | K294N | N315Q | T333S | R339K | N390D | | | | | | | | |
| 357778 | K55R | N233A | N315Q | N390D | | | | | | | | | | |
| 357779 | S49N | K294N | N315Q | V365N | N390D | A505S | | | | | | | | |
| 357780 | W50F | I118V | S198T | K294N | N315Q | N390D | | | | | | | | |
| 357781 | T127M | N315Q | N390D | A505S | | | | | | | | | | |
| 357782 | E187H | K294N | N315Q | N390D | A505S | Q518I | | | | | | | | |
| 357783 | T165Q | G171D | T180R | K294N | N315Q | N390D | | | | | | | | |
| 357784 | I108V | K294N | N315Q | R339K | | | | | | | | | | |
| 357785 | N51A | S167A | N315Q | N390D | | | | | | | | | | |
| 357786 | K286A | K294R | N315Q | N390D | A505S | | | | | | | | | |
| 357787 | A67G | K294N | N315Q | V365N | N366A | N390D | | | | | | | | |
| 357788 | T149D | T165Q | K294N | N315Q | S385N | N390D | | | | | | | | |
| 357789 | T149D | S177A | K294N | N315Q | | | | | | | | | | |
| 357790 | S26A | S177A | E187H | K294N | N315Q | N390D | | | | | | | | |
| 357791 | A67G | S198T | K294N | N315Q | N390D | Q518I | | | | | | | | |
| 357792 | W50F | A67G | S177N | K294N | N315Q | N390D | | | | | | | | |
| 357793 | N51A | E187H | S263A | K294N | N315Q | N390D | | | | | | | | |
| 357794 | A67G | E201D | K294N | N315K | N390D | | | | | | | | | |
| 357795 | S49N | A120V | A221G | K294N | N315Q | N390D | | | | | | | | |
| 357796 | K286A | N390D | | | | | | | | | | | | |
| 357797 | K294N | N315K | D360G | N390D | A507T | | | | | | | | | |
| 357798 | K105R | K294N | N315Q | S385N | A389S | N390D | | | | | | | | |
| 357799 | A102T | T149D | S167A | K294N | N315Q | N390D | | | | | | | | |
| 357800 | T149D | K294N | N315K | N390D | | | | | | | | | | |
| 357801 | K294N | D310A | N315Q | | | | | | | | | | | |
| 357802 | K55R | T232A | K294N | N315Q | S385N | N390D | | | | | | | | |
| 357803 | I108V | T180R | S198T | K294N | N315Q | N390D | | | | | | | | |
| 357804 | S26A | A120V | T127M | K294N | N315Q | N390D | | | | | | | | |
| 357805 | S49N | A67G | I118V | K294N | N315Q | N390D | | | | | | | | |
| 357806 | E103Q | K294N | N315Q | V365N | A389S | N390D | | | | | | | | |
| 357807 | K105R | S198T | A221G | K294N | N315Q | N390D | | | | | | | | |
| 357808 | S177A | T180R | K294N | N315Q | A389S | N390D | | | | | | | | |
| 357809 | S177N | K294R | N315Q | A389S | N390D | | | | | | | | | |
| 357810 | N51A | K105R | K294N | N315Q | N366A | N390D | | | | | | | | |
| 357811 | S26A | S177N | T180R | K294N | N315Q | N390D | | | | | | | | |
| 357812 | S49N | S272G | K294N | N315Q | N390D | A525P | | | | | | | | |
| 357813 | T165Q | E201D | T232A | K294N | N315Q | N390D | | | | | | | | |
| 357814 | S26A | I108V | K294N | N315Q | A389S | N390D | | | | | | | | |
| 357815 | S26A | K294N | N315Q | T333S | N390D | A507T | | | | | | | | |
| 357816 | K55R | S272G | K294N | N315Q | A389S | N390D | | | | | | | | |
| 357817 | S49N | S198T | K294N | N315Q | A389S | N390D | | | | | | | | |
| 357818 | K105R | G171D | K294N | N315Q | N390D | A525P | | | | | | | | |
| 357819 | A120V | S272G | K294N | D310A | N315Q | N390D | | | | | | | | |
| 357820 | K294N | N315K | S385N | N390D | A525P | | | | | | | | | |
| 357821 | Q126H | S263A | K294N | N315Q | V365N | N390D | | | | | | | | |
| 357822 | K294N | N315Q | R339K | A389S | N390D | Q518I | | | | | | | | |
| 357823 | W50F | N233A | K294N | D310A | N315Q | N390D | | | | | | | | |
| 357824 | T165Q | S263A | K294N | N315Q | A389S | N390D | | | | | | | | |
| 357825 | A67G | S177A | K294N | N315Q | D360G | N390D | | | | | | | | |
| 357826 | Q126H | T149D | E201D | K294N | N315Q | N390D | | | | | | | | |
| 357827 | A221G | S272G | K294N | N315Q | N390D | A505S | | | | | | | | |
| 357828 | A250R | S272G | K294N | N315Q | S385N | N390D | | | | | | | | |
| 357829 | K55R | K105R | I108V | K294N | N315Q | N390D | | | | | | | | |
| 357830 | N51A | S272G | K294N | N390D | | | | | | | | | | |
| 357831 | A102T | T165Q | K294N | N315Q | | | | | | | | | | |
| 357832 | S26A | S272G | K294N | N315K | N390D | | | | | | | | | |
| 357833 | I108V | S177N | K294N | N315Q | N390D | Q518I | | | | | | | | |
| 357834 | Q126H | S272G | K294N | N315Q | T333S | N390D | | | | | | | | |
| 357835 | T149D | S272G | K294N | N315Q | V365N | N390D | | | | | | | | |
| 357836 | E201D | S263A | K294N | N315Q | S385N | N390D | | | | | | | | |
| 357837 | A120V | E187H | A250R | K294N | N315Q | N390D | | | | | | | | |
| 357838 | S263A | K294N | D310A | N315Q | D360G | N390D | | | | | | | | |
| 357839 | S167A | T232A | K294N | N315Q | R339K | N390D | | | | | | | | |
| 357840 | E103Q | I118V | K294N | N315Q | N390D | | | | | | | | | |
| 357841 | E103Q | T127M | K294N | N315Q | D360G | N390D | | | | | | | | |
| 357842 | A67G | T232A | K294N | N315Q | A389S | N390D | | | | | | | | |
| 357843 | T149D | K294N | N315Q | A389S | N390D | A525P | | | | | | | | |
| 357844 | S26A | S49N | T149D | K294N | N315Q | N390D | | | | | | | | |
| 357845 | I118V | T180R | A250R | K294N | N315Q | N390D | | | | | | | | |
| 357846 | A67G | A250R | K294R | N315Q | N390D | | | | | | | | | |
| 357847 | G171D | S272G | K294N | N315Q | N366A | N390D | | | | | | | | |
| 372262 | A67G | Q126H | F155H | G171D | K286A | K294N | N315Q | A389S | | | | | | |
| 372263 | S26V | A67G | I118V | Q126H | G171D | K294N | D312A | N315Q | | | | | | |
| 372264 | S49N | A67G | Q126H | S130A | G171D | K294D | N315Q | | | | | | | |
| 372265 | A67G | D95A | I108V | Q126H | G171D | S198T | K294N | N315Q | | | | | | |
| 372266 | Q126H | G171D | S198T | N249A | K294N | N315Q | | | | | | | | |
| 372267 | A67G | Q126H | E288A | N315R | N366A | | | | | | | | | |
| 372268 | S26A | A67G | Q126H | S198T | E288A | K294N | N315Q | G541W | | | | | | |
| 372269 | A67G | Q126H | G171D | Q204R | K294N | N315Q | N366H | Q518I | | | | | | |
| 372270 | A67G | Q126H | G171D | E288A | K294N | I311L | N315Q | | | | | | | |
| 372271 | S26A | A67G | A102T | Q126H | G171D | K294N | N315Q | N366A | | | | | | |
| 372272 | A67G | I118V | Q126H | S198T | K294N | I311L | N315Q | A389S | | | | | | |
| 372273 | N51A | E64A | A67G | Q126H | G171D | K294N | N315Q | A389S | | | | | | |
| 372274 | A67G | Q126H | S198T | S213D | K294N | N315Q | N366A | | | | | | | |
| 372275 | S26V | S49N | A67G | Q126H | T281D | K294N | N315Q | T526D | | | | | | |
| 372276 | A67G | I118V | Q126H | G171D | N315Q | S385N | | | | | | | | |
| 372277 | A67G | I108V | I118V | Q126H | K294N | N315Q | Q518I | G541W | | | | | | |
| 372278 | S49N | A67G | Q126H | S198T | T279D | N315Q | | | | | | | | |
| 372279 | A67G | Q126H | N231A | N249S | K294N | N315Q | N366A | A389S | | | | | | |
| 372280 | S49N | A67G | A102T | Q126H | K286A | K294N | N315Q | S385N | | | | | | |
| 372281 | A67G | I118V | Q126H | S198T | N249S | K294N | N315Q | Q341A | | | | | | |
| 372282 | A67G | I118V | Q126H | E288A | K294N | N315Q | S385N | D521T | | | | | | |
| 372283 | N51A | A67G | Q126H | S198T | K294D | N315Q | S385N | | | | | | | |
| 372284 | A67G | Q126H | K294D | N315Q | R339K | Q518I | | | | | | | | |
| 372285 | I108V | I118V | Q126H | K286A | K294N | N315Q | | | | | | | | |
| 372286 | A67G | Q126H | S198T | S272G | K286A | K294N | N315Q | D521T | | | | | | |
| 372287 | A67G | Q126H | G171D | K294N | N315R | R339K | T526D | | | | | | | |
| 372288 | N51A | A67G | Q126H | G171D | S272G | K294N | N315Q | | | | | | | |
| 372289 | A67G | Q126H | D205G | K294N | D312A | N315Q | A389S | T526D | | | | | | |
| 372290 | A67G | A102T | Q126H | D205G | S263A | N315Q | | | | | | | | |
| 372291 | A67G | L94I | Q126H | S198T | S263A | K294N | N315Q | Q518I | | | | | | |
| 372292 | E64A | A67G | A102T | Q126H | K294N | N315Q | Q341A | N366H | | | | | | |
| 372293 | A67G | Q126H | F155H | S263A | T279D | K294N | N315Q | S385N | | | | | | |
| 372294 | S26A | A67G | Q126H | N249A | A292D | K294N | N315Q | | | | | | | |
| 372295 | A67G | Q126H | S272G | K294N | N315Q | Q341A | T526D | | | | | | | |
| 372296 | S26A | A67G | Q126H | K294N | N315Q | G337N | R339K | | | | | | | |
| 372297 | A67G | Q126H | N231A | A292D | K294N | D312A | N315Q | | | | | | | |
| 372298 | S26V | A67G | D205G | K294N | N315Q | | | | | | | | | |
| 372299 | A67G | Q126H | S198T | D205G | K294N | N315R | | | | | | | | |
| 372300 | A67G | I118V | Q126H | K294N | K299A | N315Q | | | | | | | | |
| 372301 | A67G | Q126H | F155H | S198T | K294N | N315Q | | | | | | | | |
| 372302 | A67G | Q126H | G171D | T279D | A292D | K294N | N315Q | | | | | | | |
| 372303 | A67G | I108V | Q126H | K294N | I311L | N315Q | N366A | | | | | | | |
| 372304 | E64A | A67G | Q126H | K286A | K294N | I311L | N315Q | | | | | | | |
| 372305 | A67G | A102T | Q126H | S198T | K294N | N315Q | A389S | | | | | | | |
| 372306 | A67G | Q126H | K286A | K294N | N315Q | R339K | N366A | | | | | | | |
| 372307 | A67G | Q126H | K294N | N315Q | R339K | S385N | A389S | | | | | | | |
| 372308 | L94I | Q126H | K294N | N315Q | R339K | | | | | | | | | |
| 372309 | E64A | A67G | Q126H | Q204R | K294N | N315Q | R339K | | | | | | | |
| 372310 | A67G | D95A | A102T | Q126H | K294N | N315Q | Q518I | | | | | | | |
| 372311 | N51A | A67G | A102T | I118V | Q126H | K294N | N315Q | | | | | | | |
| 372312 | A67G | I118V | Q126H | F155H | K294N | N315Q | G337N | | | | | | | |
| 372313 | A67G | D95A | Q126H | T279D | K294N | I311L | N315Q | | | | | | | |
| 372314 | A67G | Q126H | N249S | N315Q | G337N | | | | | | | | | |
| 372315 | A67G | L94I | I108V | Q126H | K294N | D312A | N315Q | | | | | | | |
| 372316 | A67G | Q126H | G171D | K294N | N315Q | S385N | G541W | | | | | | | |
| 372317 | A67G | Q126H | Q204R | T279D | K294N | N315Q | D521T | | | | | | | |
| 372318 | N51A | A67G | Q126H | K294N | N315Q | N366A | Q518I | | | | | | | |
| 372319 | S49N | N51A | A67G | Q126H | N249S | K294N | N315Q | | | | | | | |
| 372320 | S49N | A67G | Q126H | S213D | K294N | N315Q | A389S | | | | | | | |
| 372321 | A67G | A102T | Q126H | S213D | K294N | D312A | N315Q | | | | | | | |
| 372322 | A67G | Q126H | Q204R | K299A | N315Q | | | | | | | | | |
| 372323 | A67G | I118V | Q126H | T281D | A292D | K294N | N315Q | | | | | | | |
| 372324 | A67G | N249A | K294N | N315Q | N366A | | | | | | | | | |
| 372325 | A67G | I118V | Q126H | S272G | K294N | N315Q | N366A | | | | | | | |
| 372326 | A67G | Q126H | G171D | T281D | K294N | N315Q | G337N | | | | | | | |
| 372327 | S26V | Q126H | K294D | N315Q | | | | | | | | | | |
| 372328 | N51A | A67G | L94I | Q126H | D205G | K294N | N315Q | | | | | | | |
| 372329 | A67G | Q126H | N315Q | A389S | Q518I | | | | | | | | | |
| 372330 | A67G | Q126H | N249A | K286A | K294N | N315Q | T526D | | | | | | | |
| 372331 | A67G | I118V | Q126H | S198T | K294N | N315Q | R339K | | | | | | | |
| 372332 | S26A | A67G | I118V | Q126H | K294N | N315Q | A389S | | | | | | | |
| 372333 | A67G | D95A | K294N | N315Q | S385N | | | | | | | | | |
| 372334 | A67G | I108V | Q126H | K294N | N315Q | S385N | | | | | | | | |
| 372335 | A67G | Q126H | N231A | S272G | K294N | N315R | | | | | | | | |
| 372336 | A67G | Q126H | S130A | K294N | N315Q | S385N | Q518I | | | | | | | |
| 372337 | S26A | A67G | I108V | Q126H | S130A | K294N | N315Q | | | | | | | |
| 372338 | A67G | Q126H | N249A | S263A | K294N | N315Q | D521T | | | | | | | |
| 372339 | S26V | A67G | Q126H | S198T | K294N | N315Q | S385N | | | | | | | |
| 372340 | A67G | I108V | Q126H | F155H | S213D | K294N | N315Q | | | | | | | |
| 372341 | A67G | I118V | Q204R | K294N | N315Q | | | | | | | | | |
| 372342 | A67G | Q126H | S213D | K294N | K299A | N315Q | Q341A | | | | | | | |
| 372343 | Q126H | K294N | N315Q | N366A | S385N | | | | | | | | | |
| 372344 | A67G | Q126H | N249S | K294N | N315R | N366H | | | | | | | | |
| 372345 | A67G | Q126H | S263A | A292D | K294N | K299A | N315Q | | | | | | | |
| 372346 | A67G | I108V | Q126H | T281D | K294N | N315Q | A389S | | | | | | | |
| 372347 | E64A | A67G | Q126H | S198T | K294N | K299A | N315Q | | | | | | | |
| 372348 | A67G | L94I | Q126H | K294D | N315Q | Q341A | | | | | | | | |
| 372349 | A67G | Q126H | S130A | E288A | K294N | N315Q | G337N | | | | | | | |
| 372350 | A67G | Q126H | K286A | K294N | N315Q | N366H | G541W | | | | | | | |
| 372351 | A67G | I118V | Q126H | N231A | S263A | K294N | N315Q | | | | | | | |
| 372352 | A67G | D95A | Q126H | K294N | N315Q | A389S | | | | | | | | |
| 372353 | A67G | Q126H | S130A | S272G | K294N | N315Q | G541W | | | | | | | |
| 372354 | N51A | A67G | I108V | Q126H | N315Q | | | | | | | | | |
| 372355 | A67G | Q126H | G171D | N231A | K294N | N315Q | D521T | | | | | | | |
| 372356 | A67G | Q126H | T281D | K294N | N315Q | N366H | | | | | | | | |
| 381576 | A67G | D95A | I108V | Q126H | G171D | S198T | K294N | N315Q | | | | | | |
| 381595 | E64A | A67G | D95A | I108V | Q126H | S130A | G171D | S198T | S213D | K294N | N315Q | | | |
| 381596 | E64A | A67G | D95A | I108V | Q126H | S130A | G171D | S198T | S263A | K294N | N315Q | | | |
| 381597 | E64A | A67G | D95A | I108V | Q126H | S130A | G171D | S198T | K294N | D312A | N315Q | | | |
| 381598 | E64A | A67G | D95A | I108V | Q126H | S130A | G171D | S198T | K294N | N315Q | D521T | | | |
| 381599 | E64A | A67G | D95A | I108V | Q126H | G171D | S198T | S213D | S263A | K294N | N315Q | | | |
| 381600 | E64A | A67G | D95A | I108V | Q126H | G171D | S198T | S213D | K294N | D312A | N315Q | | | |
| 381601 | E64A | A67G | D95A | I108V | Q126H | G171D | S198T | S213D | K294N | N315Q | D521T | | | |
| 381602 | E64A | A67G | D95A | I108V | Q126H | G171D | S198T | S263A | K294N | D312A | N315Q | | | |
| 381603 | E64A | A67G | D95A | I108V | Q126H | G171D | S198T | S263A | K294N | N315Q | D521T | | | |
| 381604 | E64A | A67G | D95A | I108V | Q126H | G171D | S198T | K294N | D312A | N315Q | D521T | | | |
| 381605 | A67G | D95A | I108V | Q126H | S130A | G171D | S198T | S213D | S263A | K294N | N315Q | | | |
| 381606 | A67G | D95A | I108V | Q126H | S130A | G171D | S198T | S213D | K294N | D312A | N315Q | | | |
| 381607 | A67G | D95A | I108V | Q126H | S130A | G171D | S198T | S213D | K294N | N315Q | D521T | | | |
| 381608 | A67G | D95A | I108V | Q126H | S130A | G171D | S198T | S263A | K294N | D312A | N315Q | | | |
| 381609 | A67G | D95A | I108V | Q126H | S130A | G171D | S198T | S263A | K294N | N315Q | D521T | | | |
| 381610 | A67G | D95A | I108V | Q126H | S130A | G171D | S198T | K294N | D312A | N315Q | D521T | | | |
| 381611 | A67G | D95A | I108V | Q126H | G171D | S198T | S213D | S263A | K294N | D312A | N315Q | | | |
| 381612 | A67G | D95A | I108V | Q126H | G171D | S198T | S213D | S263A | K294N | N315Q | D521T | | | |
| 381613 | A67G | D95A | I108V | Q126H | G171D | S198T | S213D | K294N | D312A | N315Q | D521T | | | |
| 381614 | A67G | D95A | I108V | Q126H | G171D | S198T | S263A | K294N | D312A | N315Q | D521T | | | |
| 381615 | E64A | A67G | D95A | I108V | Q126H | S130A | G171D | S198T | K294N | N315Q | | | | |
| 381616 | E64A | A67G | D95A | I108V | Q126H | G171D | S198T | S213D | K294N | N315Q | | | | |
| 381617 | E64A | A67G | D95A | I108V | Q126H | G171D | S198T | S263A | K294N | N315Q | | | | |
| 381618 | E64A | A67G | D95A | I108V | Q126H | G171D | S198T | K294N | D312A | N315Q | | | | |
| 381619 | E64A | A67G | D95A | I108V | Q126H | G171D | S198T | K294N | N315Q | D521T | | | | |
| 381620 | A67G | D95A | I108V | Q126H | S130A | G171D | S198T | S213D | K294N | N315Q | | | | |
| 381621 | A67G | D95A | I108V | Q126H | S130A | G171D | S198T | S263A | K294N | N315Q | | | | |
| 381622 | A67G | D95A | I108V | Q126H | S130A | G171D | S198T | K294N | D312A | N315Q | | | | |
| 381623 | A67G | D95A | I108V | Q126H | S130A | G171D | S198T | K294N | N315Q | D521T | | | | |
| 381624 | A67G | D95A | I108V | Q126H | G171D | S198T | S213D | S263A | K294N | N315Q | | | | |
| 381625 | A67G | D95A | I108V | Q126H | G171D | S198T | S213D | K294N | D312A | N315Q | | | | |
| 381626 | A67G | D95A | I108V | Q126H | G171D | S198T | S213D | K294N | N315Q | D521T | | | | |
| 381627 | A67G | D95A | I108V | Q126H | G171D | S198T | S263A | K294N | D312A | N315Q | | | | |
| 381628 | A67G | D95A | I108V | Q126H | G171D | S198T | S263A | K294N | N315Q | D521T | | | | |
| 381629 | A67G | D95A | I108V | Q126H | G171D | S198T | K294N | D312A | N315Q | D521T | | | | |
| 381630 | E64A | A67G | D95A | I108V | Q126H | S130A | S198T | K294N | N315Q | G541W | | | | |
| 381631 | D95A | I108V | Q126H | S130A | G171D | S198T | K294N | N315Q | R339K | S506D | | | | |
| 381632 | A67G | I108V | Q126H | S130A | G171D | S198T | N249S | K294N | N315Q | D521T | | | | |
| 381633 | A67G | D95A | Q126H | S198T | S263A | N315Q | | | | | | | | |
| 381634 | E64A | A67G | D95A | I108V | Q126H | G171D | S198T | S213D | K294N | K299S | N315Q | D521T | | |
| 381635 | A67G | D95A | A102T | I108V | G171D | S198T | K294N | N315Q | A389S | G541W | | | | |
| 381636 | D95A | I108V | Q126H | G171D | S198T | N315Q | D521T | G541W | | | | | | |
| 381637 | E64A | A67G | D95A | A102S | I108V | G171D | S198T | K294N | D312A | N315Q | | | | |
| 381638 | E64A | A67G | D95A | I108V | Q126H | G171D | S198T | S263A | K294N | N315Q | A389S | D521T | | |
| 381639 | E64A | D95A | A102T | I108V | Q126H | G171D | S198T | K294N | N315Q | | | | | |
| 381640 | A67G | D95A | I108V | Q126H | G171D | S198T | S263A | K294N | K299A | N315Q | A507V | G541W | | |
| 381641 | S49N | D95A | Q126H | G171D | S198T | S213D | K294N | N315Q | | | | | | |
| 381642 | E64A | A67G | D95A | I108V | Q126H | G171D | S198T | S213D | N249S | N315Q | | | | |
| 381643 | E64A | A67G | D95A | I108V | G171D | S198T | K294N | N315Q | R339K | Y533W | | | | |
| 381644 | A67G | D95A | I108V | Q126H | G171D | S198T | S213D | K294N | D312A | N315Q | R339K | A507V | | |
| 381645 | E64A | A67G | D95A | I108V | Q126H | G171D | S198T | K294N | N315Q | D521T | V530L | N538D | | |
| 381646 | A67G | D95A | A102T | I108V | Q126H | G171D | S198T | S213D | K294N | N315Q | Q519E | A537T | | |
| 381647 | A67G | D95A | A102T | I108V | Q126H | G171D | S198T | S263A | K294N | N315Q | R339K | D521T | | |
| 381648 | D95A | I108V | Q126H | S130A | G171D | S198T | K294N | N315Q | A389S | D521T | | | | |
| 381649 | E64A | A67G | D95A | I108V | Q126H | G171D | S198T | S263A | K294N | N315Q | R339K | | | |
| 381650 | E64A | D95A | I108V | Q126H | G171D | S198T | K294N | N315Q | I516L | E522S | | | | |
| 381651 | A67G | D95A | I108V | S130A | G171D | S198T | S213D | S263A | K294N | N315Q | | | | |
| 381652 | A67G | D95A | I108V | Q126H | G171D | S198T | K294N | D312A | N315Q | D521T | G541W | | | |
| 381653 | S26A | A67G | D95A | I108V | G171D | S198T | K294N | N315Q | Q519E | D521T | | | | |
| 381654 | S26V | E64A | A67G | I108V | Q126H | G171D | S198T | K294N | N315Q | | | | | |
| 381655 | D95A | I108T | G171D | S198T | K294N | N315Q | | | | | | | | |
| 381656 | S26V | A67G | D95A | I108V | Q126H | G171D | S198T | S263A | K294N | N315Q | N538D | | | |
| 381657 | E64A | A67G | D95A | I108T | Q126H | G171D | S198T | K294N | N315Q | G541W | | | | |
| 381658 | A67G | D95A | A102T | I108V | Q126H | G171D | S198T | N315Q | N538D | | | | | |
| 381659 | A67G | D95A | I108V | Q126H | G171D | S198T | K294N | K299A | N315Q | I516L | D521T | | | |
| 381660 | A67G | D95A | Q126H | G171D | S198T | K294N | A389S | | | | | | | |
| 381661 | S26A | A67G | D95A | I108V | Q126H | S130A | G171D | S198T | K294N | N315Q | | | | |
| 381662 | A67G | D95A | I108V | Q126H | G171D | S198T | S213D | K294N | N315Q | A389S | V530L | | | |
| 381663 | A67G | D95A | I108V | Q126H | G171D | S198T | N315Q | D521T | | | | | | |
| 381664 | E64A | A67G | D95A | I108V | Q126H | S130A | G171D | S198T | N315Q | | | | | |
| 381665 | A67G | D95A | I108V | Q126H | G171D | S198T | K294N | D312A | N315Q | A389S | R542K | | | |
| 381666 | A67G | D95A | I108V | Q126H | G171D | S198T | N315Q | A537T | A539S | | | | | |
| 381667 | E64A | A67G | D95A | I108V | Q126H | G171D | S198T | K294N | N315Q | A389S | D513T | | | |
| 381668 | A67G | D95A | I108V | Q126H | G171D | S198T | K294N | N315Q | R339K | Q519E | G541W | | | |
| 381669 | A67G | D95A | A102S | I108T | Q126H | G171D | S198T | K294N | K299S | N315Q | | | | |
| 381670 | A67G | D95A | I108V | Q126H | G171D | S198T | K294N | N315Q | A389T | D521T | Y533W | | | |
| 381671 | S49N | A67G | D95A | I108V | Q126H | G171D | S198T | K294N | A537T | | | | | |
| 381672 | A67G | D95A | I108V | G171D | S198T | N315Q | A389T | | | | | | | |
| 381673 | A67G | D95A | I108V | Q126H | G171D | S198T | N315Q | R339K | A389S | | | | | |
| 381674 | A67G | I108V | Q126H | G171D | S198T | K294N | N315Q | V530L | R542K | | | | | |
| 381675 | A67G | D95A | I108V | Q126H | G171D | S198T | K294N | N315Q | A389S | A507V | | | | |
| 381676 | A67G | D95A | I108V | Q126H | S198T | K294N | K299S | N315Q | R339K | | | | | |
| 381677 | D95A | I108V | Q126H | G171D | S198T | S263A | K294N | D312A | N315Q | | | | | |
| 381678 | S26A | A67G | D95A | I108V | Q126H | G171D | S198T | K294N | A389T | | | | | |
| 381679 | A67G | D95A | I108V | Q126H | G171D | S198T | N249S | S263A | K294N | N315Q | R542K | | | |
| 381680 | A67G | D95A | I108V | G171D | S198T | K294N | N315Q | D513T | | | | | | |
| 381681 | S26V | D95A | I108V | Q126H | G171D | S198T | K294N | N315Q | D513T | | | | | |
| 381682 | A67G | D95A | A102S | I108V | Q126H | G171D | S198T | K294N | N315Q | A539S | | | | |
| 381683 | A67G | D95A | I108V | Q126H | S130A | G171D | S198T | K294N | D312A | N315Q | E522S | | | |
| 381684 | A67G | D95A | I108V | Q126H | G171D | S198T | K294N | K299A | N315Q | S506D | A539S | | | |
| 381685 | A67G | D95A | I108V | Q126H | G171D | S198T | S213D | K294N | N315Q | G541W | | | | |
| 381686 | A67G | D95A | A102T | I108V | Q126H | S130A | G171D | S198T | K294N | N315Q | I516L | | | |
| 381687 | S49N | A67G | D95A | I108V | Q126H | G171D | S198T | K294N | N315Q | R339K | D521T | | | |
| 381688 | A67G | D95A | I108V | Q126H | G171D | S198T | K294N | N315Q | S506D | Y533W | G541W | | | |
| 381689 | A67G | D95A | I108V | Q126H | G171D | S198T | S213D | K294N | N315Q | D521T | E522S | | | |
| 385020 | S26V | A67G | D95A | I108V | Q126H | G171D | S198T | S263A | K294N | N315Q | Q398H | I407V | G435A | N538D |
| 385021 | S26V | A67G | D95A | I108V | Q126H | G171D | S198T | S263A | K294N | N315Q | R404A | K472Q | T473A | N538D |
| 385022 | S26V | A67G | D95A | I108V | Q126H | G171D | S198T | S263A | K294N | N315Q | V418L | S427N | K472Q | N538D |
| 385023 | S26V | A67G | D95A | I108V | Q126H | G171D | S198T | S263A | K294N | N315Q | I407V | S427N | T483A | N538D |
| 385024 | S26V | A67G | D95A | I108V | Q126H | G171D | S198T | S263A | K294N | N315Q | Q398H | V418L | A487S | N538D |
| 385025 | S26V | A67G | D95A | I108V | Q126H | G171D | S198T | S263A | K294N | N315Q | Q398H | R404I | K472Q | N538D |
| 385026 | S26V | A67G | D95A | I108V | Q126H | G171D | S198T | S263A | K294N | N315Q | R404I | T483A | A487S | N538D |
| 385027 | S26V | A67G | D95A | I108V | Q126H | G171D | S198T | S263A | K294N | N315Q | S427N | G435A | T473A | N538D |
| 385028 | S26V | A67G | D95A | I108V | Q126H | G171D | S198T | S263A | K294N | N315Q | R404I | I407V | T498R | N538D |
| 385029 | S26V | A67G | D95A | I108V | Q126H | G171D | S198T | S263A | K294N | N315Q | R404A | G435A | T498R | N538D |
| 385030 | S26V | A67G | D95A | I108V | Q126H | G171D | S198T | S263A | K294N | N315Q | T473A | A487S | T498R | N538D |
| 385031 | S26V | A67G | D95A | I108V | Q126H | G171D | S198T | S263A | K294N | N315Q | R404A | V418L | T483A | N538D |

TABLE 2
Selected Maltotriose Amylase Variants

| GeneID | Name | Amino Acid Substitutions Relative to SEQ ID NO: 1 | | | | | | | | | | | | |
|---|---|---|---|---|---|---|---|---|---|---|---|---|---|---|
| 343612 | ETY71443 | | | | | | | | | | | | | [symbol] |
| 354034 | AmyE_R1-V7 | N181D | I297R | N366R | | | | | | | | | | [symbol] |
| 354040 | AmyE_R1-V13 | A45L | S49N | K294N | | | | | | | | | | [symbol] |
| 354041 | AmyE_R1-V14 | Q126H | G372N | S385N | | | | | | | | | | [symbol] |
| 354046 | AmyE_R1-V19 | S49N | F285Y | A389S | | | | | | | | | | [symbol] |
| 354056 | AmyE_R1-V29 | Q137G | T149D | N366A | | | | | | | | | | [symbol] |
| 354059 | AmyE_R1-V32 | T165Q | K294N | N315K | | | | | | | | | | [symbol] |
| 354118 | AmyE_R1-V91 | K294N | N315Q | N390D | | | | | | | | | | [symbol] |
| 354122 | AmyE_R1-V95 | S26A | S49N | N51A | | | | | | | | | | [symbol] |
| 357763 | AmyE_R2-V11 | S49N | G171D | K294N | N315Q | S385N | N390D | | | | | | | [symbol] |
| 357771 | AmyE_R2-V19 | A67G | Q126H | K294N | N315Q | | | | | | | | | [symbol] |
| 357775 | AmyE_R2-V23 | Q126H | K294N | D310A | N315Q | A389S | N390D | | | | | | | [symbol] |
| 357784 | AmyE_R2-V32 | I108V | K294N | N315Q | R339K | | | | | | | | | [symbol] |
| 357821 | AmyE_R2-V69 | Q126H | S263A | K294N | N315Q | V365N | N390D | | | | | | | [symbol] |
| 357826 | AmyE_R2-V74 | Q126H | T149D | E201D | K294N | N315Q | N390D | | | | | | | [symbol] |
| 357834 | AmyE_R2-V82 | Q126H | S272G | K294N | N315Q | T333S | N390D | | | | | | | [symbol] |
| 372265 | AmyE_R3-V4 | A67G | D95A | I108V | Q126H | G171D | S198T | K294N | N315Q | | | | | [symbol] |
| 372274 | AmyE_R3-V13 | A67G | Q126H | S198T | S213D | K294N | N315Q | N366A | | | | | | [symbol] |
| 372278 | AmyE_R3-V17 | S49N | A67G | Q126H | S198T | T279D | N315Q | | | | | | | [symbol] |
| 372291 | AmyE_R3-V30 | A67G | L94I | Q126H | S198T | S263A | K294N | N315Q | Q518I | | | | | [symbol] |
| 372305 | AmyE_R3-V44 | A67G | A102T | Q126H | S198T | K294N | N315Q | A389S | | | | | | [symbol] |
| 372337 | AmyE_R3-V76 | S26A | A67G | I108V | Q126H | S130A | K294N | N315Q | | | | | | [symbol] |
| 381649 | AmyE_R4_V55 | E64A | A67G | D95A | I108V | Q126H | G171D | S198T | S263A | K294N | N315Q | R339K | | [symbol] |
| 381656 | AmyE_R4_V62 | S26V | A67G | D95A | I108V | Q126H | G171D | S198T | S263A | K294N | N315Q | N538D | | [symbol] |
| 381678 | AmyE_R4_V84 | S26A | A67G | D95A | I108V | Q126H | G171D | S198T | K294N | A389T | | | | [symbol] |
| 385020 | AmyE_R4B-V1 | S26V | A67G | D95A | I108V | Q126H | G171D | S198T | S263A | K294N | N315Q | Q398H | I407V | [symbol] |
| 385022 | AmyE_R4B-V3 | S26V | A67G | D95A | I108V | Q126H | G171D | S198T | S263A | K294N | N315Q | V418L | S427N | [symbol] |
| 385023 | AmyE_R4B-V4 | S26V | A67G | D95A | I108V | Q126H | G171D | S198T | S263A | K294N | N315Q | I407V | S427N | [symbol] |
| 385027 | AmyE_R4B-V8 | S26V | A67G | D95A | I108V | Q126H | G171D | S198T | S263A | K294N | N315Q | S427N | G435A | [symbol] |

[symbol]

indicates data missing or illegible when filed

1. A variant maltotriose amylase enzyme that is a variant of a maltotriose enzyme according SEQ ID NO:1, wherein the variant comprises a Q126H amino acid substitution.

2. The variant maltotriose amylase enzyme according claim 1, wherein the variant comprises mutations selected from the group consisting of the mutations present in amino acids 30-560 of SEQ ID NOS: 2-29.

3. The maltotriose enzyme according to claim 1 wherein the variant is selected from the group consisting of the maltotriose enzymes according to amino acids 30-560 of SEQ ID NOS: 2-29.

4. The maltotriose amylase enzyme according to claim 1, wherein the maltotriose amylase enzyme is expressed from Bacillus subtilis.

5. The maltotriose amylase enzyme according to claim 2, wherein the maltotriose amylase enzyme is expressed from Bacillus subtilis.

6. The maltotriose amylase enzyme according to claim 3, wherein the maltotriose amylase enzyme is expressed from Bacillus subtilis.

7. The maltotriose amylase enzyme according to claim 4, wherein the maltotriose amylase enzyme contains a signal peptide sequence selected from the group consisting of SEQ ID NOS 30-32.

8. The maltotriose amylase enzyme according to claim 5, wherein the maltotriose amylase enzyme contains a signal peptide sequence selected from the group consisting of SEQ ID NOS 30-32.

9. The maltotriose amylase enzyme according to claim 6, wherein the maltotriose amylase enzyme contains a signal peptide sequence selected from the group consisting of SEQ ID NOS 30-32.

10. A method of making reduced sugar glucose syrup (RSGS) comprising contacting a starch with a variant of a maltotriose enzyme according SEQ ID NO:1, wherein the variant comprises a Q126H amino acid substitution at a temperature above 50° C. to form the RSGS.
